SEQUENCE LISTING (29 sequences)

SEQ ID NO: 1 - DNA, 2349 bp, Homo sapiens
  CDS (1)..(2349)
  sig_peptide (1)..(63)
atgattccctttttacccatgttttctctactattgctgcttattgttaaccctataaacgccaacaatcattatgacaagatcttggctcatagtcgtatcaggggtcgggaccaaggcccaaatgtctgtgcccttcaacagattttgggcaccaaaaagaaatacttcagcacttgtaagaactggtataaaaagtccatctgtggacagaaaacgactgtgttatatgaatgttgccctggttatatgagaatggaaggaatgaaaggctgcccagcagttttgcccattgaccatgtttatggcactctgggcatcgtgggagccaccacaacgcagcgctattctgacgcctcaaaactgagggaggagatcgagggaaagggatccttcacttactttgcaccgagtaatgaggcttgggacaacttggattctgatatccgtagaggtttggagagcaacgtgaatgttgaattactgaatgctttacatagtcacatgattaataagagaatgttgaccaaggacttaaaaaatggcatgattattccttcaatgtataacaatttggggcttttcattaaccattatcctaatggggttgtcactgttaattgtgctcgaatcatccatgggaaccagattgcaacaaatggtgttgtccatgtcattgaccgtgtgcttacacaaattggtacctcaattcaagacttcattgaagcagaagatgacctttcatcttttagagcagctgccatcacatcggacatattggaggcccttggaagagacggtcacttcacactctttgctcccaccaatgaggcttttgagaaacttccacgaggtgtcctagaaaggatcatgggagacaaagtggcttccgaagctcttatgaagtaccacatcttaaatactctccagtgttctgagtctattatgggaggagcagtctttgagacgctggaaggaaatacaattgagataggatgtgacggtgacagtataacagtaaatggaatcaaaatggtgaacaaaaaggatattgtgacaaataatggtgtgatccatttgattgatcaggtcctaattcctgattctgccaaacaagttattgagctggctggaaaacagcaaaccaccttcacggatcttgtggcccaattaggcttggcatctgctctgaggccagatggagaatacactttgctggcacctgtgaataatgcattttctgatgatactctcagcatggatcagcgcctccttaaattaattctgcagaatcacatattgaaagtaaaagttggccttaatgagctttacaacgggcaaatactggaaaccatcggaggcaaacagctcagagtcttcgtatatcgtacagctgtctgcattgaaaattcatgcatggagaaagggagtaagcaagggagaaacggtgcgattcacatattccgcgagatcatcaagccagcagagaaatccctccatgaaaagttaaaacaagataagcgctttagcaccttcctcagcctacttgaagctgcagacttgaaagagctcctgacacaacctggagactggacattatttgtgccaaccaatgatgcttttaagggaatgactagtgaagaaaaagaaattctgatacgggacaaaaatgctcttcaaaacatcattctttatcacctgacaccaggagttttcattggaaaaggatttgaacctggtgttactaacattttaaagaccacacaaggaagcaaaatctttctgaaagaagtaaatgatacacttctggtgaatgaattgaaatcaaaagaatctgacatcatgacaacaaatggtgtaattcatgttgtagataaactcctctatccagcagacacacctgttggaaatgatcaactgctggaaatacttaataaattaatcaaatacatccaaattaagtttgttcgtggtagcaccttcaaagaaatccccgtgactgtctatagacccacactaacaaaagtcaaaattgaaggtgaacctgaattcagactgattaaagaaggtgaaacaataactgaagtgatccatggagagccaattattaaaaaatacaccaaaatcattgatggagtgcctgtggaaataactgaaaaagagacacgagaagaacgaatcattacaggtcctgaaataaaatacactaggatttctactggaggtggagaaacagaagaaactctgaagaaattgttacaagaagaagacacacccgtgaggaagttgcaagccaacaaaaaagttcaaggatctagaagacgattaagggaaggtcgttctcagtga

SEQ ID NO: 2 - protein, 782 aa, Homo sapiens
MIPFLPMFSLLLLLIVNPINANNHYDKILAHSRIRGRDQGPNVCALQQILGTKKKYFSTCKNWYKKSICGQKTTVLYECCPGYMRMEGMKGCPAVLPIDHVYGTLGIVGATTTQRYSDASKLREEIEGKGSFTYFAPSNEAWDNLDSDIRRGLESNVNVELLNALHSHMINKRMLTKDLKNGMIIPSMYNNLGLFINHYPNGVVTVNCARIIHGNQIATNGVVHVIDRVLTQIGTSIQDFIEAEDDLSSFRAAAITSDILEALGRDGHFTLFAPTNEAFEKLPRGVLERIMGDKVASEALMKYHILNTLQCSESIMGGAVFETLEGNTIEIGCDGDSITVNGIKMVNKKDIVTNNGVIHLIDQVLIPDSAKQVIELAGKQQTTFTDLVAQLGLASALRPDGEYTLLAPVNNAFSDDTLSMDQRLLKLILQNHILKVKVGLNELYNGQILETIGGKQLRVFVYRTAVCIENSCMEKGSKQGRNGAIHIFREIIKPAEKSLHEKLKQDKRFSTFLSLLEAADLKELLTQPGDWTLFVPTNDAFKGMTSEEKEILIRDKNALQNIILYHLTPGVFIGKGFEPGVTNILKTTQGSKIFLKEVNDTLLVNELKSKESDIMTTNGVIHVVDKLLYPADTPVGNDQLLEILNKLIKYIQIKFVRGSTFKEIPVTVYRPTLTKVKIEGEPEFRLIKEGETITEVIHGEPIIKKYTKIIDGVPVEITEKETREERIITGPEIKYTRISTGGGETEETLKKLLQEEDTPVRKLQANKKVQGSRRRLREGRSQ

SEQ ID NO: 3 - protein, 761 aa, Homo sapiens
NNHYDKILAHSRIRGRDQGPNVCALQQILGTKKKYFSTCKNWYKKSICGQKTTVLYECCPGYMRMEGMKGCPAVLPIDHVYGTLGIVGATTTQRYSDASKLREEIEGKGSFTYFAPSNEAWDNLDSDIRRGLESNVNVELLNALHSHMINKRMLTKDLKNGMIIPSMYNNLGLFINHYPNGVVTVNCARIIHGNQIATNGVVHVIDRVLTQIGTSIQDFIEAEDDLSSFRAAAITSDILEALGRDGHFTLFAPTNEAFEKLPRGVLERIMGDKVASEALMKYHILNTLQCSESIMGGAVFETLEGNTIEIGCDGDSITVNGIKMVNKKDIVTNNGVIHLIDQVLIPDSAKQVIELAGKQQTTFTDLVAQLGLASALRPDGEYTLLAPVNNAFSDDTLSMDQRLLKLILQNHILKVKVGLNELYNGQILETIGGKQLRVFVYRTAVCIENSCMEKGSKQGRNGAIHIFREIIKPAEKSLHEKLKQDKRFSTFLSLLEAADLKELLTQPGDWTLFVPTNDAFKGMTSEEKEILIRDKNALQNIILYHLTPGVFIGKGFEPGVTNILKTTQGSKIFLKEVNDTLLVNELKSKESDIMTTNGVIHVVDKLLYPADTPVGNDQLLEILNKLIKYIQIKFVRGSTFKEIPVTVYRPTLTKVKIEGEPEFRLIKEGETITEVIHGEPIIKKYTKIIDGVPVEITEKETREERIITGPEIKYTRISTGGGETEETLKKLLQEEDTPVRKLQANKKVQGSRRRLREGRSQ

SEQ ID NO: 4 - DNA, 2352 bp, Mus musculus
  CDS (1)..(2352)
  sig_peptide (1)..(69)
atggttcctctcctgcccttatatgctctgctgctgctgttcctgtgtgatattaaccctgcaaatgccaacagttactatgacaaggtcctggctcacagccgcatcaggggtcgggatcagggcccaaacgtctgtgccctccagcaaattctgggcaccaaaaagaaatacttcagctcctgtaagaactggtatcaaggtgctatctgcgggaagaaaaccactgtgctatatgaatgctgccctggctatatgagaatggaagggatgaaaggctgccccgcagtgatgcctattgaccatgtttatggcacgctgggcattgtgggagccactaccactcagcactactccgatgtctcgaagctgagagaagagattgaaggaaaagggtcatacacgtacttcgcgccgagtaacgaggcttgggagaacctggattctgacattcgcagaggactggagaacaatgtcaatgttgagctactgaatgccttacacagccacatggttaataagagaatgttaaccaaggacctgaaacacggcatggttattccttcaatgtacaacaatctggggctttttattaaccattatcccaatggggttgtcactgtgaactgtgctcgagtcatccatgggaaccagattgccacaaatggtgtcgtccatgtcattgaccgtgtcctgacacaaattggtacctccatccaagacttccttgaagcagaagacgacctttcatcatttagagcagccgccatcacctctgacctcttggagtcccttggaagagatggtcacttcacgctctttgctcccaccaatgaagctttcgagaaactgccacgaggtgtcctagaaaggatcatgggagacaaagtggcttctgaagctctcatgaagtaccacatcctaaataccctccagtgctctgaggccatcactggaggagccgtgtttgagaccatggaaggaaacactattgagatagggtgcgaaggggacagtatctccattaacggaatcaagatggtgaacaagaaagacattgtgactaagaatggtgtcatccacctgattgatgaagtcctcattcctgattctgccaaacaagttattgagctggctggaaaacagcaaaccactttcaccgacctggtagcccaattaggcttggcatcctctctgaagccagatggagagtacaccttattagcacctgtgaacaatgcgttctctgatgacactctgagcatggaccaacgccttcttaagctaattctgcaaaatcacatattgaaagtaaaagttggccttagcgacctctacaatggacagatactggaaaccattggaggcaaacaactccgagtctttgtgtatcggacggctatctgcatagaaaactcatgcatggtgagaggaagcaagcagggaaggaatggtgccattcacatattccgagaaatcatccaaccagcagagaaatccctgcacgacaagctgcggcaagacaagcgctttagcatcttcctcagcctccttgaagctgcagatttgaaagatctcctgacacagcccggagattggaccttgtttgcaccaaccaatgatgccttcaagggaatgactagcgaagaaagggagcttctgattggggataaaaatgctctccaaaacatcattctttatcacctgaccccaggggtttatattggaaagggattcgaacccggagtcactaatatcctgaagaccacacagggaagcaaaatctatctgaaaggagtaaacgaaacgcttctagtgaatgagttgaagtccaaagaatctgacatcatgacgacaaatggtgtcatccacgtcgtggacaaactcctctatccagcagatattccagttggaaatgatcagctcttggaattactgaacaaactgataaaatacatccaaatcaagtttgttcgtggcagcaccttcaaagaaatccccatgactgtctatagacctgcaatgacgaagatccaaattgaaggtgatcccgacttcaggctgattaaagaaggcgaaacggtgacagaagtgatccacggagagccagtcattaaaaagtacaccaaaatcatagatggagttcctgttgaaataactgaaaaacagactcgggaagaacgaatcattacaggtcctgagataaaatataccaggatttccacaggaggtggagaaacaggagagaccttgcagaaattcttgcaaaaagacacacctgcaaagaagataccagccaacaaaagggttcaagggcctagaagacgatcaagagaaggccgttctcagtga

SEQ ID NO: 5 - protein, 783 aa, Mus musculus
MVPLLPLYALLLLFLCDINPANANSYYDKVLAHSRIRGRDQGPNVCALQQILGTKKKYFSSCKNWYQGAICGKKTTVLYECCPGYMRMEGMKGCPAVMPIDHVYGTLGIVGATTTQHYSDVSKLREEIEGKGSYTYFAPSNEAWENLDSDIRRGLENNVNVELLNALHSHMVNKRMLTKDLKHGMVIPSMYNNLGLFINHYPNGVVTVNCARVIHGNQIATNGVVHVIDRVLTQIGTSIQDFLEAEDDLSSFRAAAITSDLLESLGRDGHFTLFAPTNEAFEKLPRGVLERIMGDKVASEALMKYHILNTLQCSEAITGGAVFETMEGNTIEIGCEGDSISINGIKMVNKKDIVTKNGVIHLIDEVLIPDSAKQVIELAGKQQTTFTDLVAQLGLASSLKPDGEYTLLAPVNNAFSDDTLSMDQRLLKLILQNHILKVKVGLSDLYNGQILETIGGKQLRVFVYRTAICIENSCMVRGSKQGRNGAIHIFREIIQPAEKSLHDKLRQDKRFSIFLSLLEAADLKDLLTQPGDWTLFAPTNDAFKGMTSEERELLIGDKNALQNIILYHLTPGVYIGKGFEPGVTNILKTTQGSKIYLKGVNETLLVNELKSKESDIMTTNGVIHVVDKLLYPADIPVGNDQLLELLNKLIKYIQIKFVRGSTFKEIPMTVYRPAMTKIQIEGDPDFRLIKEGETVTEVIHGEPVIKKYTKIIDGVPVEITEKQTREERIITGPEIKYTRISTGGGETGETLQKFLQKDTPAKKIPANKRVQGPRRRSREGRSQ

SEQ ID NO: 6 - protein, 760 aa, Mus musculus
NSYYDKVLAHSRIRGRDQGPNVCALQQILGTKKKYFSSCKNWYQGAICGKKTTVLYECCPGYMRMEGMKGCPAVMPIDHVYGTLGIVGATTTQHYSDVSKLREEIEGKGSYTYFAPSNEAWENLDSDIRRGLENNVNVELLNALHSHMVNKRMLTKDLKHGMVIPSMYNNLGLFINHYPNGVVTVNCARVIHGNQIATNGVVHVIDRVLTQIGTSIQDFLEAEDDLSSFRAAAITSDLLESLGRDGHFTLFAPTNEAFEKLPRGVLERIMGDKVASEALMKYHILNTLQCSEAITGGAVFETMEGNTIEIGCEGDSISINGIKMVNKKDIVTKNGVIHLIDEVLIPDSAKQVIELAGKQQTTFTDLVAQLGLASSLKPDGEYTLLAPVNNAFSDDTLSMDQRLLKLILQNHILKVKVGLSDLYNGQILETIGGKQLRVFVYRTAICIENSCMVRGSKQGRNGAIHIFREIIQPAEKSLHDKLRQDKRFSIFLSLLEAADLKDLLTQPGDWTLFAPTNDAFKGMTSEERELLIGDKNALQNIILYHLTPGVYIGKGFEPGVTNILKTTQGSKIYLKGVNETLLVNELKSKESDIMTTNGVIHVVDKLLYPADIPVGNDQLLELLNKLIKYIQIKFVRGSTFKEIPMTVYRPAMTKIQIEGDPDFRLIKEGETVTEVIHGEPVIKKYTKIIDGVPVEITEKQTREERIITGPEIKYTRISTGGGETGETLQKFLQKDTPAKKIPANKRVQGPRRRSREGRSQ

SEQ ID NO: 7 - DNA, 2352 bp, Rattus norvegicus
  CDS (1)..(2352)
  sig_peptide (1)..(69)
atggttcctctcctgcccttatctgctctgctgctgctgttcctgtgtgacgttgaccccgcaaatgccaacagttactatgacaaggtcctagctcacagccgcatcaggggtcgggatcagggcccaaatgtctgtgccctccagcagattctgggcaccaaaaagaaatacttcagctcctgtaagaactggtatcaaggtgctatctgcgggaagaaaaccactgtgctatatgaatgctgccccggctatatgagaatggaagggatgaaaggctgcccagcagtgatgcccattgaccatgtttatggcacgctgggcatcgtgggagccacgaccactcaacactattctgatgtctcgaagctcagggaagagattgaaggaaaagggtcctacacatacttcgcgccgagtaacgaagcttgggacaacctggattccgacatccgcagaggactagagaacaatgtcaatgttgagttactgaacgctttacacagccacatggttaataagagaatgctaaccaaggacctgaaacacggcatggttattccttcaatgtacaacaatctggggctttttatcaatcattatcccaatggggttgtcactgtgaactgtgctcgagtaatccacgggaaccagattgccacaaatggtgttgtccatgtcatcgaccgtgtcctgacacaaattggcacctccatccaagacttcattgaagcagaagatgagctttcatcattcagagcggctgccatcacttctgaccttttggagtcccttggaagagacggtcacttcacactctttgctcccaccaatgaggctttcgagaaactcccacgaggagtcctagaaaggatcatgggagacaaagtggcttctgaagctctcatgaagtaccacatcctgaataccctccagtgctctgaggctatcacaggaggagcggtgtttgagaccatggaaggaaacactattgaaatagggtgtgagggagacagcatctccattaacggaatcaagatggtgaacaagaaagacattgtgacgaagaatggtgtcatccacctgattgatgaagtcctcattcctgattctgctaaacaagttattgagctggctggaaaacagcaaaccactttcacggacctggtagcccagttagggttggcgtcttctctgaagccggatggagagtacacgctgttagcgcctgtgaacaatgcgttctctgatgacactctgagcatggaccagcgccttcttaagctaattctgcaaaatcacatattgaaagtaaaagtcggccttagtgatctctacaatggacagattctggagaccattggaggcaaacaactccgtgtcttcgtgtatcggacggctatctgcatagaaaactcatgcatggtgagaggaagcaagcaggggaggaacggtgccattcacatattccgagagatcatccaaccggcggagaagtccctgcacgaaaaactgcgccaagataagcgcttcagcatcttcctcagcctcctcgaagctgcagatctgaaagatcttctgacacagcccggagattggaccttgtttgcaccaaccaatgatgccttcaagggaatgactaatgaagaaagggagattctgattggggataaaaatgctctccaaaacatcattctttaccacctgaccccaggggtttatattggaaagggatttgaacccggagtcaccaacatcctgaagaccacacagggaagcaaaatctatgtgaaaggagtcaatgagacgcttttggtgaatgagttgaagtccaaagaatctgacatcatgacaacaaacggcgtcattcacgttgtggacaaactcctctatccagcagacattccggttggaaatgatcagctcttggaattactgaacaaactgataaaatacatccaaattaagttcgttcgtggcagcaccttcaaagaaatccccatgactgtctatagacctgcaatgacgaagatccacattgaaggcgagcctgacttcaggctgattaaagaaggtgaaacagtgacagaagtgatccacggagaaccagtcattaaaaagtacaccaaaatcatagacggggttcctgttgaaataactgaaaaagagacccgggaagaacgcatcatcacaggtcctgagataaaatacactaggatttccacaggaggtggggaaacagaagagaccctgcagaaattcttgcaaaaagacacacctgcaaagaagatacaagccaacaaaagggttcaagggtctagaaggcgatcaagagaaggccgttctcagtga

SEQ ID NO: 8 - protein, 783 aa, Rattus norvegicus
MVPLLPLSALLLLFLCDVDPANANSYYDKVLAHSRIRGRDQGPNVCALQQILGTKKKYFSSCKNWYQGAICGKKTTVLYECCPGYMRMEGMKGCPAVMPIDHVYGTLGIVGATTTQHYSDVSKLREEIEGKGSYTYFAPSNEAWDNLDSDIRRGLENNVNVELLNALHSHMVNKRMLTKDLKHGMVIPSMYNNLGLFINHYPNGVVTVNCARVIHGNQIATNGVVHVIDRVLTQIGTSIQDFIEAEDELSSFRAAAITSDLLESLGRDGHFTLFAPTNEAFEKLPRGVLERIMGDKVASEALMKYHILNTLQCSEAITGGAVFETMEGNTIEIGCEGDSISINGIKMVNKKDIVTKNGVIHLIDEVLIPDSAKQVIELAGKQQTTFTDLVAQLGLASSLKPDGEYTLLAPVNNAFSDDTLSMDQRLLKLILQNHILKVKVGLSDLYNGQILETIGGKQLRVFVYRTAICIENSCMVRGSKQGRNGAIHIFREIIQPAEKSLHEKLRQDKRFSIFLSLLEAADLKDLLTQPGDWTLFAPTNDAFKGMTNEEREILIGDKNALQNIILYHLTPGVYIGKGFEPGVTNILKTTQGSKIYVKGVNETLLVNELKSKESDIMTTNGVIHVVDKLLYPADIPVGNDQLLELLNKLIKYIQIKFVRGSTFKEIPMTVYRPAMTKIHIEGEPDFRLIKEGETVTEVIHGEPVIKKYTKIIDGVPVEITEKETREERIITGPEIKYTRISTGGGETEETLQKFLQKDTPAKKIQANKRVQGSRRRSREGRSQ

SEQ ID NO: 9 - protein, 760 aa, Rattus norvegicus
NSYYDKVLAHSRIRGRDQGPNVCALQQILGTKKKYFSSCKNWYQGAICGKKTTVLYECCPGYMRMEGMKGCPAVMPIDHVYGTLGIVGATTTQHYSDVSKLREEIEGKGSYTYFAPSNEAWDNLDSDIRRGLENNVNVELLNALHSHMVNKRMLTKDLKHGMVIPSMYNNLGLFINHYPNGVVTVNCARVIHGNQIATNGVVHVIDRVLTQIGTSIQDFIEAEDELSSFRAAAITSDLLESLGRDGHFTLFAPTNEAFEKLPRGVLERIMGDKVASEALMKYHILNTLQCSEAITGGAVFETMEGNTIEIGCEGDSISINGIKMVNKKDIVTKNGVIHLIDEVLIPDSAKQVIELAGKQQTTFTDLVAQLGLASSLKPDGEYTLLAPVNNAFSDDTLSMDQRLLKLILQNHILKVKVGLSDLYNGQILETIGGKQLRVFVYRTAICIENSCMVRGSKQGRNGAIHIFREIIQPAEKSLHEKLRQDKRFSIFLSLLEAADLKDLLTQPGDWTLFAPTNDAFKGMTNEEREILIGDKNALQNIILYHLTPGVYIGKGFEPGVTNILKTTQGSKIYVKGVNETLLVNELKSKESDIMTTNGVIHVVDKLLYPADIPVGNDQLLELLNKLIKYIQIKFVRGSTFKEIPMTVYRPAMTKIHIEGEPDFRLIKEGETVTEVIHGEPVIKKYTKIIDGVPVEITEKETREERIITGPEIKYTRISTGGGETEETLQKFLQKDTPAKKIQANKRVQGSRRRSREGRSQ

SEQ ID NO: 10 - DNA, 2511 bp, Homo sapiens
  CDS (1)..(2511)
  sig_peptide (1)..(63)
atgattccctttttacccatgttttctctactattgctgcttattgttaaccctataaacgccaacaatcattatgacaagatcttggctcatagtcgtatcaggggtcgggaccaaggcccaaatgtctgtgcccttcaacagattttgggcaccaaaaagaaatacttcagcacttgtaagaactggtataaaaagtccatctgtggacagaaaacgactgttttatatgaatgttgccctggttatatgagaatggaaggaatgaaaggctgcccagcagttttgcccattgaccatgtttatggcactctgggcatcgtgggagccaccacaacgcagcgctattctgacgcctcaaaactgagggaggagatcgagggaaagggatccttcacttactttgcaccgagtaatgaggcttgggacaacttggattctgatatccgtagaggtttggagagcaacgtgaatgttgaattactgaatgctttacatagtcacatgattaataagagaatgttgaccaaggacttaaaaaatggcatgattattccttcaatgtataacaatttggggcttttcattaaccattatcctaatggggttgtcactgttaattgtgctcgaatcatccatgggaaccagattgcaacaaatggtgttgtccatgtcattgaccgtgtgcttacacaaattggtacctcaattcaagacttcattgaagcagaagatgacctttcatcttttagagcagctgccatcacatcggacatattggaggcccttggaagagacggtcacttcacactctttgctcccaccaatgaggcttttgagaaacttccacgaggtgtcctagaaaggttcatgggagacaaagtggcttccgaagctcttatgaagtaccacatcttaaatactctccagtgttctgagtctattatgggaggagcagtctttgagacgctggaaggaaatacaattgagataggatgtgacggtgacagtataacagtaaatggaatcaaaatggtgaacaaaaaggatattgtgacaaataatggtgtgatccatttgattgatcaggtcctaattcctgattctgccaaacaagttattgagctggctggaaaacagcaaaccaccttcacggatcttgtggcccaattaggcttggcatctgctctgaggccagatggagaatacactttgctggcacctgtgaataatgcattttctgatgatactctcagcatggttcagcgcctccttaaattaattctgcagaatcacatattgaaagtaaaagttggccttaatgagctttacaacgggcaaatactggaaaccatcggaggcaaacagctcagagtcttcgtatatcgtacagctgtctgcattgaaaattcatgcatggagaaagggagtaagcaagggagaaacggtgcgattcacatattccgcgagatcatcaagccagcagagaaatccctccatgaaaagttaaaacaagataagcgctttagcaccttcctcagcctacttgaagctgcagacttgaaagagctcctgacacaacctggagactggacattatttgtgccaaccaatgatgcttttaagggaatgactagtgaagaaaaagaaattctgatacgggacaaaaatgctcttcaaaacatcattctttatcacctgacaccaggagttttcattggaaaaggatttgaacctggtgttactaacattttaaagaccacacaaggaagcaaaatctttctgaaagaagtaaatgatacacttctggtgaatgaattgaaatcaaaagaatctgacatcatgacaacaaatggtgtaattcatgttgtagataaactcctctatccagcagacacacctgttggaaatgatcaactgctggaaatacttaataaattaatcaaatacatccaaattaagtttgttcgtggtagcaccttcaaagaaatccccgtgactgtctatacaactaaaattataaccaaagttgtggaaccaaaaattaaagtgattgaaggcagtcttcagcctattatcaaaactgaaggacccacactaacaaaagtcaaaattgaaggtgaacctgaattcagactgattaaagaaggtgaaacaataactgaagtgatccatggagagccaattattaaaaaatacaccaaaatcattgatggagtgcctgtggaaataactgaaaaagagacacgagaagaacgaatcattacaggtcctgaaataaaatacactaggatttctactggaggtggagaaacagaagaaactctgaagaaattgttacaagaagaggtcaccaaggtcaccaaattcattgaaggtggtgatggtcatttatttgaagatgaagaaattaaaagactgcttcagggagacacacccgtgaggaagttgcaagccaacaaaaaagttcaaggttctagaagacgattaagggaaggtcgttctcagtga

SEQ ID NO: 11 - protein, 836 aa, Homo sapiens
MIPFLPMFSLLLLLIVNPINANNHYDKILAHSRIRGRDQGPNVCALQQILGTKKKYFSTCKNWYKKSICGQKTTVLYECCPGYMRMEGMKGCPAVLPIDHVYGTLGIVGATTTQRYSDASKLREEIEGKGSFTYFAPSNEAWDNLDSDIRRGLESNVNVELLNALHSHMINKRMLTKDLKNGMIIPSMYNNLGLFINHYPNGVVTVNCARIIHGNQIATNGVVHVIDRVLTQIGTSIQDFIEAEDDLSSFRAAAITSDILEALGRDGHFTLFAPTNEAFEKLPRGVLERFMGDKVASEALMKYHILNTLQCSESIMGGAVFETLEGNTIEIGCDGDSITVNGIKMVNKKDIVTNNGVIHLIDQVLIPDSAKQVIELAGKQQTTFTDLVAQLGLASALRPDGEYTLLAPVNNAFSDDTLSMVQRLLKLILQNHILKVKVGLNELYNGQILETIGGKQLRVFVYRTAVCIENSCMEKGSKQGRNGAIHIFREIIKPAEKSLHEKLKQDKRFSTFLSLLEAADLKELLTQPGDWTLFVPTNDAFKGMTSEEKEILIRDKNALQNIILYHLTPGVFIGKGFEPGVTNILKTTQGSKIFLKEVNDTLLVNELKSKESDIMTTNGVIHVVDKLLYPADTPVGNDQLLEILNKLIKYIQIKFVRGSTFKEIPVTVYTTKIITKVVEPKIKVIEGSLQPIIKTEGPTLTKVKIEGEPEFRLIKEGETITEVIHGEPIIKKYTKIIDGVPVEITEKETREERIITGPEIKYTRISTGGGETEETLKKLLQEEVTKVTKFIEGGDGHLFEDEEIKRLLQGDTPVRKLQANKKVQGSRRRLREGRSQ

SEQ ID NO: 12 - DNA, 2517 bp, Mus musculus
  CDS (1)..(2517)
  sig_peptide (1)..(69)
atggttcctctcctgcccttatatgctctgctgctgctgttcctgtgtgatattaaccctgcaaatgccaacagttactatgacaaggtcctggctcacagccgcatcaggggtcgggatcagggcccaaacgtctgtgccctccagcaaattctgggcaccaaaaagaaatacttcagctcctgtaagaactggtatcaaggtgctatctgcgggaagaaaaccactgtgctatatgaatgctgccctggctatatgagaatggaagggatgaaaggctgccccgcagtgatgcctattgaccatgtttatggcacgctgggcattgtgggagccactaccactcagcactactccgatgtctcgaagctgagagaagagattgaaggaaaagggtcatacacgtacttcgcgccgagtaacgaggcttgggagaacctggattctgacattcgcagaggactggagaacaatgtcaatgttgagctactgaatgccttacacagccacatggttaataagagaatgttaaccaaggacctgaaacacggcatggttattccttcaatgtacaacaatctggggctttttattaaccattatcccaatggggttgtcactgtgaactgtgctcgagtcatccatgggaaccagattgccacaaatggtgtcgtccatgtcattgaccgtgtcctgacacaaattggtacctccatccaagacttccttgaagcagaagacgacctttcatcatttagagcagccgccatcacctctgacctcttggagtcccttggaagagatggtcacttcacgctctttgctcccaccaatgaagctttcgagaaactgccacgaggtgtcctagaaaggatcatgggagacaaagtggcttctgaagctctcatgaagtaccacatcctaaataccctccagtgctctgaggccatcactggaggagccgtgtttgagaccatggaaggaaacactattgagatagggtgcgaaggggacagtatctccattaacggaatcaagatggtgaacaagaaagacattgtgactaagaatggtgtcatccacctgattgatgaagtcctcattcctgattctgccaaacaagttattgagctggctggaaaacagcaaaccactttcaccgacctggtagcccaattaggcttggcatcctctctgaagccagatggagagtacaccttattagcacctgtgaacaatgcgttctctgatgacactctgagcatggaccaacgccttcttaagctaattctgcaaaatcacatattgaaagtaaaagttggccttagcgacctctacaatggacagatactggaaaccattggaggcaaacaactccgagtctttgtgtatcggacggctatctgcatagaaaactcatgcatggtgagaggaagcaagcagggaaggaatggtgccattcacatattccgagaaatcatccaaccagcagagaaatccctgcacgacaagctgcggcaagacaagcgctttagcatcttcctcagcctccttgaagctgcagatttgaaagatctcctgacacagcccggagattggaccttgtttgcaccaaccaatgatgccttcaagggaatgactagcgaagaaagggagcttctgattggggataaaaatgctctccaaaacatcattctttatcacctgaccccaggggtttatattggaaagggattcgaacccggagtcactaatatcctgaagaccacacagggaagcaaaatctatctgaaaggagtaaacgaaacgcttctagtgaatgagttgaagtccaaagaatctgacatcatgacgacaaatggtgtcatccacgtcgtggacaaactcctctatccagcagatattccagttggaaatgatcagctcttggaattactgaacaaactgataaaatacatccaaatcaagtttgttcgtggcagcaccttcaaagaaatccccatgactgtctatacaactaaaattataaccaaagtcgtggaaccaaaaattaaagtcattcaaggcagtcttcagcctattatcaaaacggaaggacctgcaatgacgaagatccaaattgaaggtgatcccgacttcaggctgattaaagaaggcgaaacggtgacagaagtgatccacggagagccagtcattaaaaagtacaccaaaatcatagatggagttcctgttgaaataactgaaaaacagactcgggaagaacgaatcattacaggtcctgagataaaatataccaggatttccacaggaggtggagaaacaggagagaccttgcagaaattcttgcaaaaagaggtctccaaggtcacaaagttcattgaaggtggcgatggtcacttatttgaagatgaggagattaaaagactgcttcagggagacacacctgcaaagaagataccagccaacaaaagggttcaagggcctagaagacgatcaagagaaggccgttctcagtga

SEQ ID NO: 13 - protein, 838 aa, Mus musculus
MVPLLPLYALLLLFLCDINPANANSYYDKVLAHSRIRGRDQGPNVCALQQILGTKKKYFSSCKNWYQGAICGKKTTVLYECCPGYMRMEGMKGCPAVMPIDHVYGTLGIVGATTTQHYSDVSKLREEIEGKGSYTYFAPSNEAWENLDSDIRRGLENNVNVELLNALHSHMVNKRMLTKDLKHGMVIPSMYNNLGLFINHYPNGVVTVNCARVIHGNQIATNGVVHVIDRVLTQIGTSIQDFLEAEDDLSSFRAAAITSDLLESLGRDGHFTLFAPTNEAFEKLPRGVLERIMGDKVASEALMKYHILNTLQCSEAITGGAVFETMEGNTIEIGCEGDSISINGIKMVNKKDIVTKNGVIHLIDEVLIPDSAKQVIELAGKQQTTFTDLVAQLGLASSLKPDGEYTLLAPVNNAFSDDTLSMDQRLLKLILQNHILKVKVGLSDLYNGQILETIGGKQLRVFVYRTAICIENSCMVRGSKQGRNGAIHIFREIIQPAEKSLHDKLRQDKRFSIFLSLLEAADLKDLLTQPGDWTLFAPTNDAFKGMTSEERELLIGDKNALQNIILYHLTPGVYIGKGFEPGVTNILKTTQGSKIYLKGVNETLLVNELKSKESDIMTTNGVIHVVDKLLYPADIPVGNDQLLELLNKLIKYIQIKFVRGSTFKEIPMTVYTTKIITKVVEPKIKVIQGSLQPIIKTEGPAMTKIQIEGDPDFRLIKEGETVTEVIHGEPVIKKYTKIIDGVPVEITEKQTREERIITGPEIKYTRISTGGGETGETLQKFLQKEVSKVTKFIEGGDGHLFEDEEIKRLLQGDTPAKKIPANKRVQGPRRRSREGRSQ

SEQ ID NO: 14 - DNA, 618 bp, Homo sapiens
  CDS (1)..(618)
gacacacctgttggaaatgatcaactgctggaaatacttaataaattaatcaaatacatccaaattaagtttgttcgtggtagcaccttcaaagaaatccccgtgactgtctatacaactaaaattataaccaaagttgtggaaccaaaaattaaagtgattgaaggcagtcttcagcctattatcaaaactgaaggacccacactaacaaaagtcaaaattgaaggtgaacctgaattcagactgattaaagaaggtgaaacaataactgaagtgatccatggagagccaattattaaaaaatacaccaaaatcattgatggagtgcctgtggaaataactgaaaaagagacacgagaagaacgaatcattacaggtcctgaaataaaatacactaggatttctactggaggtggagaaacagaagaaactctgaagaaattgttacaagaagaggtcaccaaggtcaccaaattcattgaaggtggtgatggtcatttatttgaagatgaagaaattaaaagactgcttcagggagacacacccgtgaggaagttgcaagccaacaaaaaagttcaaggatctagaagacgattaagggaaggtcgttctcagtga

SEQ ID NO: 15 - protein, 205 aa, Homo sapiens
DTPVGNDQLLEILNKLIKYIQIKFVRGSTFKEIPVTVYTTKIITKVVEPKIKVIEGSLQPIIKTEGPTLTKVKIEGEPEFRLIKEGETITEVIHGEPIIKKYTKIIDGVPVEITEKETREERIITGPEIKYTRISTGGGETEETLKKLLQEEVTKVTKFIEGGDGHLFEDEEIKRLLQGDTPVRKLQANKKVQGSRRRLREGRSQ

SEQ ID NO: 16 - DNA, 30 bp, Artificial Sequence
  Description of Artificial Sequence Primer P1F
gataaaatacatccaaatcaagtttgttcg

SEQ ID NO: 17 - DNA, 28 bp, Artificial Sequence
  Description of Artificial Sequence Primer P1R
cgtggatcacttctggtcaccgtttcgc

SEQ ID NO: 18 - DNA, 25 bp, Artificial Sequence
  Description of Artificial Sequence Primer P2F
ctgaaaaacagactcgggaagaacg

SEQ ID NO: 19 - DNA, 24 bp, Artificial Sequence
  Description of Artificial Sequence Primer P2R
aaactctgtggtctggcctctggg

SEQ ID NO: 20 - DNA, 30 bp, Artificial Sequence; identical to SEQ ID NO: 16
  Description of Artificial Sequence Primer P3F

SEQ ID NO: 21 - DNA, 24 bp, Artificial Sequence
  Description of Artificial Sequence Primer P3R
aaactctgtggtctggcctctggg

SEQ ID NO: 22 - DNA, 20 bp, Artificial Sequence
  Description of Artificial Sequence Primer gapdhF
actttgtcaagctcatttcc

SEQ ID NO: 23 - DNA, 20 bp, Artificial Sequence
  Description of Artificial Sequence Primer gapdhR
tgcagcgaactttarrgctg

SEQ ID NO: 24 - DNA, 21 bp, Artificial Sequence
  Description of Artificial Sequence Primer PGK-R
ctaaagcgcatgctccagact

SEQ ID NO: 25 - DNA, 25 bp, Artificial Sequence
  Description of Artificial Sequence Primer Peri-R4
gcacctgcctcttcccaattacagg

SEQ ID NO: 26 - DNA, 25 bp, Artificial Sequence
  Description of Artificial Sequence Primer Wild-F
gttcttacagaaagcagaaggatac

SEQ ID NO: 27 - DNA, 25 bp, Artificial Sequence
  Description of Artificial Sequence Primer Wild-R
ttaaatcactccacagcagaacacg

SEQ ID NO: 28 - DNA, 25 bp, Artificial Sequence
  Description of Artificial Sequence Primer Nock-F
catgatagcttctctcccagttctc

SEQ ID NO: 29 - DNA, 25 bp, Artificial Sequence
  Description of Artificial Sequence Primer Nock-R
cttgcaataagtaaaacagctcccc

(19) United States
(12) Patent Application Publication
(10) Pub. No.: US 2011/0130335 A1
(43) Pub. Date: Jun. 2, 2011

(54) THERAPEUTIC AGENT AND TEST AGENT FOR DISEASE WITH MYOCARDIAL NECROSIS

(75) Inventor: Akira Kudo, Kanagawa (JP)

(73) Assignee: MitsubishiChemical Medience Corporation, Minato-ku, Tokyo (JP)

(21) Appl. No.: 13/054,782

(22) PCT Filed: Dec. 15, 2008
(86) PCT No.: PCT/JP2008/072743

§ 371 Date: Jan. 18, 2011

(30) Foreign Application Priority Data
Jul. 18, 2008 (JP) 2008-187851

(51) Int. Cl. A61K 38/16; C12Q 1/68; C12Q 1/02; G01N 33/566; A61K 31/7088; C07K 14/435; C07K 16/18; C07H 21/04

(52) U.S. Cl. 514/16.4; 435/6.1; 435/29; 436/501; 514/44 R; 530/350; 530/387.9; 536/23.5

(57) ABSTRACT

Disclosed are a medicament for treating or preventing diseases with myocardial necrosis, which contains, as an active ingredient, a periostin variant (ΔbΔe) polypeptide or a modified form thereof or a polynucleotide encoding the same; and a diagnostic agent for diseases with myocardial necrosis containing a DNA encoding a periostin variant (ΔbΔe) polypeptide or a partial fragment thereof or an antibody which specifically recognizes a periostin variant (ΔbΔe) polypeptide.

### TECHNICAL FIELD

The present invention relates to a medicament and a diagnostic agent for treating diseases with myocardial necrosis, which contain a periostin variant (ΔbΔe) protein or a gene (DNA or RNA) encoding the protein. Furthermore, it relates to a screening method for a medicament for treating diseases with myocardial necrosis, which uses a periostin variant (ΔbΔe) protein or a gene (DNA or RNA) encoding the protein, and to a medicament that can be obtained by the screening method.

### BACKGROUND ART

Myocardial infarction is one life-style disease related posing one of the greatest problems in developed countries.

Myocardial infarction is a disease having high onset rate and fatality rate. In Japan, it is estimated that about 150000 persons develop acute myocardial infarction annually, about 30% of which are dead. The recovery rate of patients with heart failure is still bad, and establishment of efficient treating methods has been demanded. Furthermore, the detailed mechanism of how the tissue with myocardial infarction is healed has been unclear.

Periostin is a protein that is thought to play an important role in a mechanism of regenerating the bone on the stimulus by gravity or load (Non-patent Document 1). Furthermore, when a human histological tissue was comprehensively examined by using a human periostin antibody, it was found that periostin was generated also in human or mouse tissue with myocardial infarction (Non-patent Document 2).

The Non-patent Document 1 reports the presence of several types of variants lacking periostin at the C-terminal side. Furthermore, it is suggested that periostin plays an important role in the recruitment and adhesion of osteoblast. However, there is neither consideration nor suggestion as to the relationship between any periostin variants and the function.

The Patent Document 1 reports the presence of several types of variants lacking periostin at the C-terminal side in several types of normal tissue. Furthermore, it is reported that the expression level of periostin is extremely high in patients with breast cancer having bone metastasis, or preeclampsia, various lung cancers. However, there is neither consideration nor suggestion as to the relationship between any periostin variants and the function in cancer or for diagnostic use.

On the other hand, the Non-patent Document 3 reports that cardiac rupture easily occurs in periostin-lacking mice. Then, Non-patent Document 4 reports that when periostin protein is allowed to react with a rat cardiomyocyte, the cardiomyocyte is increased. Thus, it is anticipated that periostin plays an important role in the heart. However, the role of periostin variants, in which several types are thought to be present, is not indicated.
[Patent Document 1] Japanese Translation Publication (Kohyo) No. 2005-500059
[Non-patent document 1] Journal of bone and mineral research (US), 1999, Vol. 14, No. 7, p. 1239-1249
[Non-patent document 2] Archives of oral biology (UK), 2005, Vol. 50, No. 12, p. 1023-1031
[Non-patent document 3] Circulation research (US), 2007, Vol. 101, No. 3, p. 313-321
[Non-patent document 4] Nature medicine (US), 2007, Vol. 13, No. 8, p. 962-969

### DISCLOSURE OF THE INVENTION

### Problems to be Solved by the Invention

If the prevention of cardiac rupture and/or the healing of an infarct region after myocardial infarction can be promoted, amelioration and recovery from myocardial infarction can be advanced, resulting in reduction of the mortality. Identification of molecules having such activities and development of a drug and a diagnostic agent for ameliorating a cardiac function using the molecule have been demanded. This is thought to be effective in myocarditis and cardiomyopathy (for example, dilated cardiomyopathy) accompanying myocardial necrosis such as one occurring in the infarct region.

That is to say, a problem to be solved by the present invention is to provide a material for promoting the prevention of cardiac rupture and/or the healing of a myocardial necrosis region after myocardial infarction or well-known diseases (myocardial infarction, myocarditis, cardiomyopathy, and the like) accompanying myocardial necrosis as a medicament and a diagnostic agent for treating myocardial infarction.

### Means for Solving the Problems

In order to solve the above-mentioned problems, the present inventors have keenly studied, focused on periostin that is an extracellular matrix protein, and clarified a part of the mechanism.

Firstly, when the expression of periostin in the heart was examined, a periostin protein was expressed in the heart in the mouse development process but the expression disappeared in an adult mouse myocardium. Next, a myocardial infarction model mouse was created by ligating the left ventricle descending coronary artery. From three days after the ligation, expression of the periostin protein was found in the border site between the myocardium and an infarct layer. On day 7, expression in the myocardial infarct layer was rapidly increased. It is reported that periostin has a large number of splicing variants, and the present inventors have investigated them. Consequently, it has been clear that in the acute stage of the myocardial infarction, isoform called ΔbΔe [hereinafter, referred to as “periostin variant (ΔbΔe) protein,” and also referred to as a periostin variant (ΔbΔe) polypeptide, a periostin variant (ΔbΔe), and ΔbΔe], which lacks a part of the C-terminal, is specifically expressed.

Furthermore, in order to investigate a role of the periostin at the onset of myocardial infarction, the present inventors generated periostin gene-defective mice. In the periostin gene-defective mice, no cardiac abnormality in function and morphology has been found until at least 16 weeks old. However, when acute myocardial infarction was induced in the mice, the infarct healing ability in the acute stage was low, and cardiac rupture occurred remarkably frequently (68.1%) as compared with wild-type mice (31.3%). Furthermore, when the stiffness of a myocardium was examined, although no difference between the wild-type mice and the defective mice was found under the physiological conditions, the stiffness in a site with the myocardial infarction was remarkably reduced in the defective mice. On the other hand, in the chronic state, ventricle dilation was suppressed. From the above-mention, although the defective of periostin does not affect the cardiac morphology formation or the cardiac function under the physiological conditions, it is shown that periostin plays an important role in healing of infarction and ventricle remodeling after myocardial infarction.

When the cause thereof was examined, in the myocardial infarct region of the periostin defective mice, the deposition of type I collagen and fibronectin as an extracellular substrate protein was reduced, collagen fibril formation was also immature, and furthermore, the number itself of vimentin/αSMA (α smooth muscle actin) positive cardiac fibroblast as a production source of the extracellular substrate was reduced. Furthermore, in order to confirm whether such a phenomenon of the myocardial infarct region in periostin defective mice can be directly recovered by periostin, when a ΔbΔe gene was introduced into the infarct region, the number of the αSMA-positive fibroblast in the infarct region was increased, thus enabling the development frequency of the cardiac rupture to be improved. From these results, it was thought that periostin plays an important role in cell movement of cardiac fibroblast to the infarct region and collagen fibril formation.

Since it is said that phosphorylation of FAK (Focal Adhesion Kinase) is important for cell movement, the present inventors focused on the phosphorylation in a myocardial infarct region in periostin-defective mice. Then, interestingly, it is shown that at the time of infarction, the phosphorylation of FAK is largely reduced in periostin-defective mice as compared with the wild-type mice. In order to investigate the relationship between periostin and the FAK in more detail, the investigation was carried out by adding ΔbΔe under culture conditions. As a result, when ΔbΔe was added, the phosphorylation of FAK was shown to be accelerated. However, by inhibiting αV integrin, phosphorylation of FAK and the migration of fibroblast induced by periostin were inhibited. From the experiment results, it is shown that the signal transmission mechanism related to periostin is associated with αV integrin or FAK, and the movement of fibroblast to the infarct region and activation by periostin and collagen fibril formation are important in healing of acute myocardial infarction.

As mentioned in the above-mentioned findings, the present inventors have found that a periostin variant (ΔbΔe) has a specific activity in healing of the infarct region, and then completed the present invention. That is to say, the present invention provides the following (1) to (42).

(1) A medicament for treating or preventing diseases with myocardial necrosis, comprising a periostin variant (ΔbΔe) polypeptide as an active ingredient.
(2) A medicament for treating or preventing diseases with myocardial necrosis, comprising, as an active ingredient:

[a] a polypeptide comprising the amino acid sequence of SEQ ID NO: 3, SEQ ID NO: 6, or SEQ ID NO: 9;

[b] a polypeptide comprising an amino acid sequence in which one or several amino acids are deleted, substituted or added in the amino acid sequence of SEQ ID NO: 3, SEQ ID NO: 6, or SEQ ID NO: 9, and having an activity of healing a myocardial necrosis site; or

[c] a polypeptide comprising an amino acid sequence that has an identity of not less than 60% to the amino acid sequence of SEQ ID NO: 3, SEQ ID NO: 6, or SEQ ID NO: 9, and having an activity of healing a myocardial necrosis site.

(3) The medicament according to (1) or (2), wherein the medicament has an effect of ameliorating a cardiac function.
(4) The medicament according to (1) to (3), wherein the medicament has an effect of promoting cardiac regeneration.
(5) A method for treating or preventing diseases with myocardial necrosis, the method comprising administering a therapeutically effective amount of periostin variant (ΔbΔe) polypeptide to a subject to be treated.
(6) A method for treating or preventing diseases with myocardial necrosis, the method comprising administering a therapeutically effective amount of polypeptide to a subject to be treated, the polypeptide being selected from the group consisting of:

[a] a polypeptide comprising the amino acid sequence of SEQ ID NO: 3, SEQ ID NO: 6, or SEQ ID NO: 9;

[b] a polypeptide comprising an amino acid sequence in which one or several amino acids are deleted, substituted or added in the amino acid sequence of SEQ ID NO: 3, SEQ ID NO: 6, or SEQ ID NO: 9, and having an activity of healing a myocardial necrosis site; or

[c] a polypeptide comprising an amino acid sequence that has an identity of not less than 60% to the amino acid sequence of SEQ ID NO: 3, SEQ ID NO: 6, or SEQ ID NO: 9, and having an activity of healing a myocardial necrosis site.

(7) The method according to (5) or (6), wherein the method has an effect of ameliorating a cardiac function.
(8) The method according to (5) to (7), wherein the method has an effect of promoting cardiac regeneration.
(9) A use of a periostin variant (ΔbΔe) polypeptide in manufacture of a medicament for treating diseases with myocardial necrosis.
(10) A use of the following [a], [b] or [c] in manufacture of a medicament for treating diseases with myocardial necrosis:

[a] a polypeptide comprising the amino acid sequence of SEQ ID NO: 3, SEQ ID NO: 6, or SEQ ID NO: 9;

[b] a polypeptide comprising an amino acid sequence in which one or several amino acids are deleted, substituted or added in the amino acid sequence of SEQ ID NO: 3, SEQ ID NO: 6, or SEQ ID NO: 9, and having an activity of healing a myocardial necrosis site; or

[c] a polypeptide comprising an amino acid sequence that has an identity of not less than 60% to the amino acid sequence of SEQ ID NO: 3, SEQ ID NO: 6, or SEQ ID NO: 9, and having an activity of healing a myocardial necrosis site.

(11) The use according to (9) or (10), wherein the medicament has an effect of ameliorating a cardiac function.
(12) The use according to (9) to (11), wherein the medicament has an effect of promoting cardiac regeneration.
(13) A medicament for treating or preventing diseases with myocardial necrosis, comprising a polynucleotide encoding a periostin variant (ΔbΔe).
(14) A medicament for treating or preventing diseases with myocardial necrosis, comprising:

[a] a polynucleotide encoding a polypeptide comprising the amino acid sequence of SEQ ID NO: 3, SEQ ID NO: 6, or SEQ ID NO: 9;

[b] a polynucleotide encoding a polypeptide comprising an amino acid sequence in which one or several amino acids are deleted, substituted or added in the amino acid sequence of SEQ ID NO: 3, SEQ ID NO: 6, or SEQ ID NO: 9, and having an activity of healing a myocardial necrosis site;

[c] a polynucleotide encoding a polypeptide comprising an amino acid sequence having an identity of not less than 60% to the amino acid sequence of SEQ ID NO: 3, SEQ ID NO: 6, or SEQ ID NO: 9 and having an activity of healing a myocardial necrosis site; or

[d] a polynucleotide comprising a nucleotide sequence of SEQ ID NO: 1, SEQ ID NO: 4, or SEQ ID NO: 7.

(15) The medicament according to (13) or (14), wherein the medicament has an effect of ameliorating a cardiac function.
(16) The medicament according to (13) to (15), wherein the medicament has an effect of promoting cardiac regeneration.
(17) A method for treating or preventing diseases with myocardial necrosis, the method comprising administering a therapeutically effective amount of polynucleotide encoding a periostin variant (ΔbΔe) to a subject to be treated.
(18) A method for treating or preventing diseases with myocardial necrosis, the method comprising administering a therapeutically effective amount of polynucleotide to a subject to be treated, the polynucleotide being selected from the group consisting of:

[a] a polynucleotide encoding a polypeptide comprising the amino acid sequence of SEQ ID NO: 3, SEQ ID NO: 6, or SEQ ID NO: 9;

[b] a polynucleotide encoding a polypeptide comprising an amino acid sequence in which one or several amino acids are deleted, substituted or added in the amino acid sequence of SEQ ID NO: 3, SEQ ID NO: 6, or SEQ ID NO: 9, and having an activity of healing a myocardial necrosis site;

[c] a polynucleotide encoding a polypeptide comprising an amino acid sequence having an identity of not less than 60% to the amino acid sequence of SEQ ID NO: 3, SEQ ID NO: 6, or SEQ ID NO: 9 and having an activity of healing a myocardial necrosis site; or

[d] a polynucleotide comprising a nucleotide sequence of SEQ ID NO: 1, SEQ ID NO: 4, or SEQ ID NO: 7.

(19) The method according to (17) or (18), wherein the method has an effect of ameliorating a cardiac function.
(20) The method according to (17) to (19), wherein the method has an effect of promoting cardiac regeneration.
(21) A use of a polynucleotide encoding a periostin variant (ΔbΔe) in manufacture of a medicament for treating diseases with myocardial necrosis.
(22) A use of the following [a], [b] or [c] in manufacture of a medicament for treating diseases with myocardial necrosis:

[a] a polynucleotide encoding a polypeptide comprising the amino acid sequence of SEQ ID NO: 3, SEQ ID NO: 6, or SEQ ID NO: 9;

[b] a polynucleotide encoding a polypeptide comprising an amino acid sequence in which one or several amino acids are deleted, substituted or added in the amino acid sequence of SEQ ID NO: 3, SEQ ID NO: 6, or SEQ ID NO: 9, and having an activity of healing a myocardial necrosis site;

[c] a polynucleotide encoding a polypeptide comprising an amino acid sequence having an identity of not less than 60% to the amino acid sequence of SEQ ID NO: 3, SEQ ID NO: 6, or SEQ ID NO: 9 and having an activity of healing a myocardial necrosis site; or

[d] a polynucleotide comprising a nucleotide sequence of SEQ ID NO: 1, SEQ ID NO: 4, or SEQ ID NO: 7.

(23) The use according to (21) or (22), wherein the medicament has an effect of ameliorating a cardiac function.
(24) The use according to (21) to (23), wherein the medicament has an effect of promoting cardiac regeneration.
(25) A diagnostic agent for diseases with myocardial necrosis, comprising:

[a] a DNA encoding a periostin variant (ΔbΔe) polypeptide, or a partial fragment thereof;

[b] a DNA consisting of a nucleotide sequence of SEQ ID NO: 1, SEQ ID NO: 4, or SEQ ID NO: 7, or a partial fragment thereof; or

[c] an oligonucleotide having a sequence consisting of consecutive 5 to 60 nucleotides in the nucleotide sequence of SEQ ID NO: 1, SEQ ID NO: 4 or SEQ ID NO: 7, or a derivative thereof, and an oligonucleotide having a sequence complementary to the oligonucleotide, or a derivative thereof.

(26) A diagnostic agent for diseases with myocardial necrosis, comprising an antibody specifically recognizing a periostin variant (ΔbΔe) polypeptide.
(27) A diagnostic method for diseases with myocardial necrosis, the method comprising the steps of:

(1) preparing a DNA or cDNA derived from a test specimen from a biological sample obtained from a subject to be tested;

(2) detecting a mutation of a DNA encoding a periostin variant (ΔbΔe) polypeptide in the DNA or cDNA derived from a test specimen, by using:

[a] the DNA encoding the periostin variant (ΔbΔe) polypeptide, or a partial fragment thereof;

[b] a DNA consisting of a nucleotide sequence of SEQ ID NO: 1, SEQ ID NO: 4, or SEQ ID NO: 7, or a partial fragment thereof; or

[c] an oligonucleotide having a sequence consisting of consecutive 5 to 60 nucleotides in the nucleotide sequence of SEQ ID NO: 1, SEQ ID NO: 4 or SEQ ID NO: 7, or a derivative thereof, and an oligonucleotide having a sequence complementary to the oligonucleotide, or a derivative thereof, and

(3) determining a risk, a type, a degree and/or a state of a disease with myocardial necrosis based on the mutation.

(28) A diagnostic method for diseases with myocardial necrosis, the method comprising the steps of:

(1) preparing a DNA or cDNA derived from a test specimen from a biological sample obtained from a subject to be tested;

(2) specifically amplifying a DNA encoding a periostin variant (ΔbΔe) polypeptide in the DNA or cDNA derived from the test specimen, and analyzing an expression level thereof, by using:

[a] a DNA encoding a periostin variant (ΔbΔe) polypeptide, or a partial fragment thereof;

[b] a DNA consisting of nucleotide sequence of SEQ ID NO: 1, SEQ ID NO: 4, or SEQ ID NO: 7, or a partial fragment thereof; or

[c] an oligonucleotide having a sequence consisting of consecutive 5 to 60 nucleotides in the nucleotide sequence of SEQ ID NO: 1, SEQ ID NO: 4 or SEQ ID NO: 7, or a derivative thereof, and an oligonucleotide having a sequence complementary to the oligonucleotide, or a derivative thereof, and

(3) determining a degree and/or a state of a disease with myocardial necrosis based on the expression level.

(29) A diagnostic method for diseases with myocardial necrosis, the method comprising the steps of:

(1) preparing a specimen from a biological sample obtained from a subject to be tested;

(2) detecting an expression level and/or a structural change of a periostin variant (ΔbΔe) polypeptide in the specimen by using an antibody specifically recognizing the periostin variant (ΔbΔe) polypeptide, and

(3) determining a risk, a cause, a degree, and/or a state of a disease with myocardial necrosis based on the expression level and/or the structural change.

(30) A screening method for a medicament for treating or preventing diseases with myocardial necrosis, wherein (i) an expression level of the polypeptide in a cell expressing the polypeptide described in (1) or (2) and (ii) an expression level of the polypeptide when the cell expressing the polypeptide is brought into contact with a testing material are compared with each other so as to select a material for increasing the expression level of the polypeptide.
(31) A screening method for a medicament for treating or preventing diseases with myocardial necrosis, wherein (i) a function of a cell expressing the polypeptide described in (1) or (2) and (ii) a function of the cell when the cell expressing the polypeptide is brought into contact with a testing material are compared with each other so as to select a material having an activity of controlling the function of the cell.
(32) The screening method according to (30) or (31), wherein the cell is a cell lacking a gene encoding periostin.
(33) The method according to (30) to (32), wherein the medicament for treating or preventing diseases with myocardial necrosis is a material having an effect of ameliorating a cardiac function.
(34) The method according to (30) to (32), wherein the medicament for treating or preventing diseases with myocardial necrosis is a material having an effect of promoting cardiac regeneration.
(35) A compound obtained by the methods according to (30) to (34), or a pharmacologically acceptable salt thereof.
(36) A medicament for treating or preventing diseases with myocardial necrosis, comprising the compound or the pharmacologically acceptable salt thereof according to (35).
(37) The medicament for treating or preventing diseases with myocardial necrosis according to (36), wherein the medicament has an effect of ameliorating a cardiac function.
(38) The medicament for treating or preventing diseases with myocardial necrosis according to (36), wherein the medicament has an effect of healing and promoting cardiac regeneration.
(39) A method for treating or preventing diseases with myocardial necrosis, the method comprising administering a therapeutically effective amount of the compound or the pharmacologically acceptable salt thereof according to (35) to a subject to be treated.
(40) A use of the compound or the pharmacologically acceptable salt thereof according to (35) in manufacture of a medicament for treating diseases with myocardial necrosis.
(41) An antibody specifically recognizing a periostin variant (ΔbΔe) protein.
(42) An immunological detection or a quantitation method of a periostin variant (ΔbΔe) protein, which uses the antibody according to (41).

Another aspect of the present invention provides a method for treating diseases with myocardial necrosis, which includes administering a therapeutically effective amount of the polypeptide described in the above-mentioned (1) or (2) to mammalian including humans.

A further aspect of the present invention provides a use of the above-mentioned polypeptide in manufacture of a medicament for treating diseases with myocardial necrosis.

A further aspect of the present invention provides a method for treating diseases with myocardial necrosis, which includes administering a therapeutically effective amount of polynucleotide (preferably, DNA) described in the above-mentioned (6) to mammalian including humans.

A further aspect of the present invention provides a use of the above-mentioned polynucleotide (preferably, DNA) in manufacture of a gene therapy agent for treating diseases with myocardial necrosis.

### Effect of the Invention

The present invention provides a therapeutic agent for diseases with myocardial necrosis, which contains a periostin variant (ΔbΔe) protein as an active ingredient, a therapeutic agent for diseases with myocardial necrosis, which contains a DNA encoding the protein as an active ingredient, a screening method for a therapeutic agent for diseases with myocardial necrosis using the protein or the DNA, and a diagnostic agent, a diagnostic method and a monitoring method for diseases with myocardial necrosis, which use an antibody specifically recognizing the DNA or the protein.

### BRIEF DESCRIPTION OF THE DRAWINGS

FIG. 1 is a view schematically showing a full-length structure of periostin, and each structure of various splicing variants in a CT domain at the C-terminal, and illustrating an expression level of each variant after acute myocardial infarction.

FIG. 2 is a view illustrating a method for deleting exon 1 of a periostin gene to create a periostin knockout mouse.

FIG. 3 is a graph showing an effect of a periostin variant (ΔbΔe) protein or full-length periostin in a periostin knockout mouse (periostin−/−) with acute myocardial infarction. The line “a” shows a result in mice to which an Ad-nlsLacZ vector was administered (control), the line “b” shows a result in mice to which an Ad-ΔbΔe virus was administered, and the line “c” shows a result in mice to which an Ad-Full virus was administered.

FIG. 4 is a view showing a nucleotide sequence (SEQ ID NO: 14) and an amino acid sequence (SEQ ID NO: 15) of a CT domain of a human full-length periostin and the locations of regions a1 to f2.

### BEST MODES FOR CARRYING OUT THE INVENTION

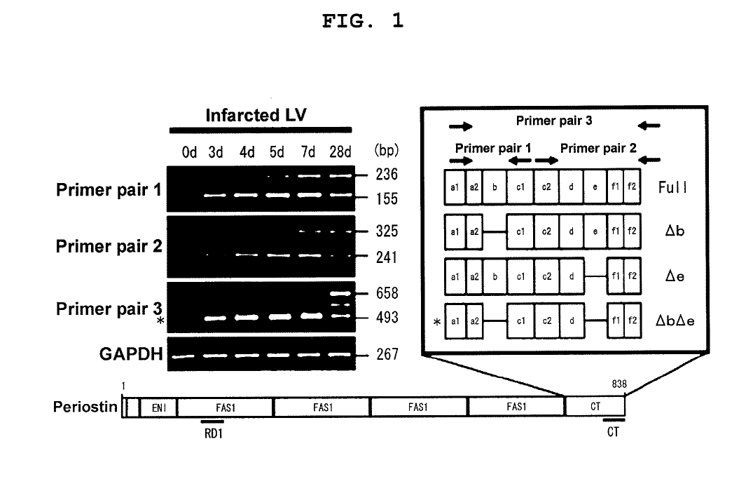

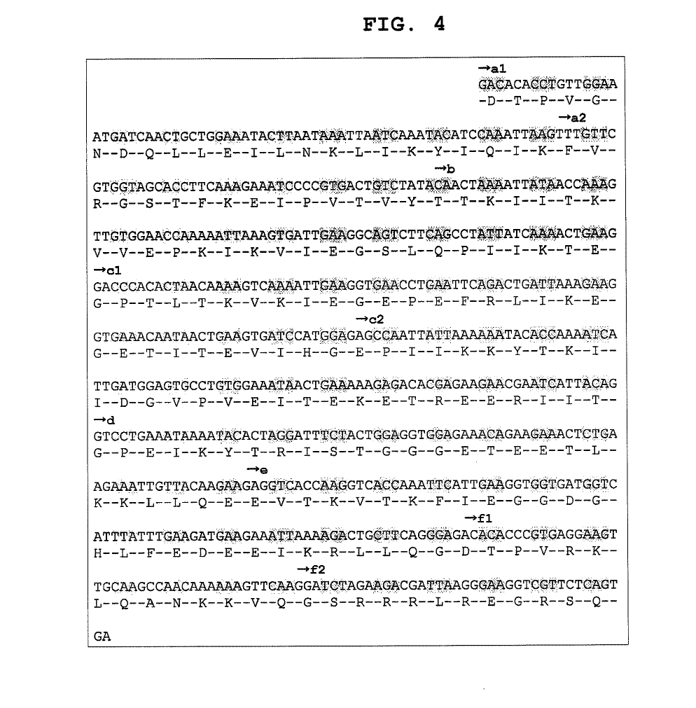

Proteins contained in a medicament of the present invention may include natural periostin variant (ΔbΔe) proteins or the modified proteins thereof. A CT domain located at the C-terminal of periostin includes a regional a1 (nucleotide number 1 to 69), a region a2 (nucleotide number 70 to 115), a region b (nucleotide number 116 to 196), a region c1 (nucleotide number 197 to 286), a region c2 (nucleotide number 287 to 376), a region d (nucleotide number 377 to 454), a region e (nucleotide number 455 to 538), a region f1 (nucleotide number 539 to 579), and a region f2 (nucleotide number 580 to 615) as shown in FIG. 1 (mouse periostin) or FIG. 4 (human periostin). Note here that the numeric values shown in each parenthesis following each region is a nucleotide number in the nucleotide sequence (FIG. 4 and SEQ ID NO: 14) encoding the CT domain of human periostin. As a variant at the C-terminal of periostin, a periostin variant (Δb) protein in which the region b is removed by splicing, a periostin variant (Δe) protein in which the region e is removed by splicing, and a periostin variant (ΔbΔe) protein in which the region b and region e are removed by splicing, and the like, are known.

The periostin variant (ΔbΔe) protein or the modified protein thereof may be a naturally occurring protein or a protein produced by genetic engineering technique as long as it is a protein having an activity of healing a myocardial necrosis site. Examples of the naturally occurring proteins may include periostin variant (ΔbΔe) proteins derived from any mammalians such as human, monkey, pig, cow, sheep, horse, rat, and mouse. Note here that the naturally occurring periostin variant (ΔbΔe) protein is translated as a precursor having a signal sequence at the N-terminal, but in the present invention, a precursor or a mature form in which a signal sequence is cleaved can be used.

Specific examples of the periostin variant (ΔbΔe) proteins may include a human periostin variant (ΔbΔe) protein of SEQ ID NO: 2 (precursor) or SEQ ID NO: 3 (mature form), a mouse periostin variant (ΔbΔe) protein of SEQ ID NO: 5 (precursor) or SEQ ID NO: 6 (mature form), and a rat periostin variant (ΔbΔe) protein of SEQ ID NO: 8 (precursor) or SEQ ID NO: 9 (mature form).

Furthermore, the periostin variant (ΔbΔe) proteins or the modified proteins thereof contained in a medicament of the present invention may include:

[a] a polypeptide comprising the amino acid sequence of SEQ ID NO: 3, SEQ ID NO: 6, or SEQ ID NO: 9 (preferably, a polypeptide comprising the amino acid sequence of SEQ ID NO: 3, SEQ ID NO: 6, or SEQ ID NO: 9, and having an activity of healing a myocardial necrosis site, and more preferably, a polypeptide consisting of the amino acid sequence of SEQ ID NO: 3, SEQ ID NO: 6, or SEQ ID NO: 9);

[b] a polypeptide comprising an amino acid sequence in which one or several amino acids have been deleted, substituted or added in the amino acid sequence of SEQ ID NO: 3, SEQ ID NO: 6, or SEQ ID NO: 9 (preferably, consisting of an amino acid sequence in which one or several amino acids have been deleted, substituted or added in the amino acid sequence of SEQ ID NO: 3, SEQ ID NO: 6, or SEQ ID NO: 9), and having an activity of healing a myocardial necrosis site; or

[c] a polypeptide comprising an amino acid sequence having an identity to the amino acid sequence of SEQ ID NO: 3, SEQ ID NO: 6, or SEQ ID NO: 9 of not less than 60% and having an activity of healing a myocardial necrosis site (preferably, a polypeptide consisting of an amino acid sequence having an identity to the amino acid sequence of SEQ ID NO: 3, SEQ ID NO: 6, or SEQ ID NO: 9 of not less than 60%).

Examples of the polypeptide comprising the amino acid sequence of SEQ ID NO: 3, SEQ ID NO: 6, or SEQ ID NO: 9 may include a fusion protein in which various polypeptide sequences or oligopeptide sequences are added to the amino acid sequence of SEQ ID NO: 3, SEQ ID NO: 6, or SEQ ID NO: 9. Examples of the fusion proteins may include a fusion protein to which maltose binding protein, oligohistidine, an antibody peptide epitope, a human immunoglobulin constant region, protein A, a signal sequence, and the like, are bound. Furthermore, the fusion protein can be provided with a sequence that can be specifically cleaved by a well-known technique.

A polypeptide that forms a polypeptide comprising an amino acid sequence in which one or several amino acids have been deleted, substituted or added in the amino acid sequence of SEQ ID NO: 3, SEQ ID NO: 6, or SEQ ID NO: 9, and having an activity of healing a myocardial necrosis site can be obtained by introducing, for example, site-specific mutation into the polypeptide consisting of the amino acid sequence of SEQ ID NO: 3, SEQ ID NO: 6, or SEQ ID NO: 9 by using the site-specific mutation introducing method described in Molecular Cloning, A Laboratory Manual, Second Edition, Cold Spring Harbor Laboratory Press (1989) (hereinafter, abbreviated as Molecular Cloning, Vol. 2), Current Protocols in Molecular Biology, John Wiley & Sons (1987-1997) (hereinafter, abbreviated as Current Protocols in Molecular Biology), Nucleic Acids Research, 10, 6487 (1982), Proc. Natl. Acad. Sci. USA, 79, 6409 (1982), Gene, 34, 315 (1985), Nucleic Acids Research, 13, 4431 (1985), Proc. Natl. Acad. Sci. USA, 82, 488 (1985), and the like.

The number of amino acids to be deleted, substituted, or added may be one or more, and the upper limit is not particularly defined. However, it is the number of amino acids that can be deleted, substituted and/or added by the well-known methods such as the above-mentioned site-specific mutation method, and it is one to several tens, preferably 1 to 20, more preferably 1 to 10, and further more preferably 1 to 5.

The phrase one or more amino acid residues have been deleted, substituted or added in the amino acid sequence of a protein or polypeptide contained in a medicament of the present invention means that deletion, substitution, and/or addition of one or a plurality of amino acid residues are included in the position of any one or a plurality of the amino acid sequences in the same sequence. The deletion, substitution, and/or addition may occur concurrently. Amino acid residues to be substituted or added may be natural or non-natural amino acid residues. Examples of the natural amino acid residues may include L-alanine, L-asparagine, L-asparatic acid, L-glutamine, L-glutamic acid, glycine, L-histidine, L-isoleucine, L-leucine, L-lysine, L-arginine, L-methionine, L-phenyl alanine, L-proline, L-serine, L-threonine, L-tryptophane, L-tyrosine, L-valine, L-cysteine, and the like.

The following are examples of amino acid residues that can be mutually substituted. The amino acid residues belonging to the same group can be mutually substituted.

Group A: leucine, isoleucine, norleucine, valine, norvaline, alanine, 2-amino butanoic acid, methionine, O-methylserine, t-butylglycine, t-butylalanine, cyclohexylalanine.

Group B: asparatic acid, glutamic acid, isoasparatic acid, isoglutamic acid, 2-aminoadipic acid, 2-aminosuberic acid.

Group C: asparagine, glutamine.

Group D: lysine, arginine, ornithine, 2,4-diamino butanoic acid, 2,3-diaminopropionic acid.

Group E: proline, 3-hydroxyproline, 4-hydroxyproline.

Group F: serine, threonine, homoserine.

Group G: phenyl alanine, tyrosine.

It is preferable that the polypeptide comprising an amino acid sequence having an identity to the amino acid sequence of SEQ ID NO: 3, SEQ ID NO: 6, or SEQ ID NO: 9 of not less than 60% and having an activity of healing a myocardial necrosis site has at least not less than 60%, generally not less than 80%, preferably not less than 90%, more preferably not less than 95%, furthermore preferably not less than 98%, and particularly preferably not less than 99% identity to the amino acid sequence of SEQ ID NO: 3, SEQ ID NO: 6, or SEQ ID NO: 9.

The identity of amino acid sequences or nucleotide sequences can be determined by using algorithm BLAST by Karlin and Altschul [Pro. Natl. Acad. Sci. USA, 90, 5873 (1993)] and FASTA [Methods Enzymol., 183, 63 (1990)]. Based on the algorithm BLAST, programs called BLASTN and BLASTX are developed [J. Mol. Biol., 215, 403 (1990)]. When a nucleotide sequence is analyzed based on BLAST and by BLASTN, the parameters are set to, for example, Score=100 and wordlength=12. Furthermore, when an amino acid sequence is analyzed based on BLAST and by using BLASTX, the parameters are set to, for example, score=50 and wordlength=3. When BLAST and Gapped BLAST program are used, default parameter of each program is used. Specific techniques of these analysis methods are well known (http://www.ncbi.nlm.nih.gov.).

A method for confirming whether or not a polypeptide contained in the medicament of the present invention has an activity of preventing cardiac rupture and/or healing a myocardial necrosis site can be carried out by using the technique of the well-known myocardial infarction model except that a periostin knockout animal in which a periostin gene has been knocked out is used, although the confirmation is not limited to this method. For example, as mentioned in the following Example, acute myocardial infarction can be induced by ligating the left ventricle descending coronary artery (LAD ligation; Michael, 1995 Am J Physiol Heart Circ Physiol. 269:H2147-2154). A polypeptide that is subjected to confirmation of the above-mentioned activity (hereinafter, referred to as “polypeptide to be evaluated”), or a gene encoding the polypeptide is administered to a periostin knockout animal, myocardial infarction is developed by the above-mentioned technique, and then the state of the infarct region (myocardial necrosis portion) is observed by visual observation or based on well-known indicators, whereby it is possible to confirm whether or not the polypeptide has the activity of healing the myocardial necrosis site. Note here that the polypeptide to be evaluated or a gene may be administered before the onset of myocardial infarction or after (or concurrently with) the onset of myocardial infarction.

As a specific indicator for healing an infarct region, introgression using virus, or healing of a myocardial infarction site by administration of periostin protein can be considered based on, for example, the number of αSMA (α smooth muscle actin) positive cells, the amount of collagen and the degree of cross-linking, and the degree of healing of tissue by a histological analysis, or based on the survival rate after myocardial infarction. For example, when the survival rate after myocardial infarction is used as the indicator, an activity of healing myocardial necrosis site is determined to be present when the survival rate one week after myocardial infarction is not less than 40% (preferably not less than 50%).

In a medicament of the present invention, in addition to a periostin variant (ΔbΔe) protein or the modified protein thereof, for example, a periostin variant (Δb) protein, a periostin variant (Δe) protein, or full-length periostin (Full) protein may be contained. The periostin variant (Δb) protein, the periostin variant (Δe) protein, or full-length periostin (Full) protein may be a naturally occurring protein or a protein produced by a genetic engineering technique as long as the protein has an activity of healing a myocardial necrosis site. Examples of the naturally occurring proteins may include periostin proteins derived from any mammalians such as human, monkey, pig, cow, sheep, horse, rat, and mouse, as well as a periostin variant protein.

Specific examples of the full-length periostin (Full) protein may include human full-length periostin (Full) protein of SEQ ID NO: 10 (precursor) or SEQ ID NO: 11 (mature form), and a mouse full-length periostin (Full) protein of SEQ ID NO: 12 (precursor) or SEQ ID NO: 13 (mature form).

Hereinafter, the present invention is further described by taking cases in which a periostin variant (ΔbΔe) protein is used as the specific embodiments of the polypeptide used in the present invention as an example, but the present invention is not necessarily limited to these embodiments, and the invention can be carried out by using the above-mentioned various polypeptides.

1. Method for Obtaining Periostin Variant (ΔbΔe) Protein

When the full-length cDNA is well-known like a periostin variant (ΔbΔe), an appropriate length DNA fragment including a portion encoding the protein may be prepared if necessary based on the full-length cDNA. Alternatively, cDNA encoding human a periostin variant (ΔbΔe) can be isolated by screening the cDNA library derived from, for example, human placenta or lung based on the sequence information of the full-length cDNA. When the obtained cDNA is not the full-length cDNA, the full-length cDNA can be obtained by screening of the cDNA library or a RACE method [rapid amplification of cDNA ends; Frohman M A et al., Proc. Natl. Acad. Sci. USA, 85, 8998 (1988)] by using a clone of the cDNA as a probe.

The nucleotide sequence of the obtained cDNA clone is determined by using, for example, a DNA sequencer, the nucleotide sequence is translated into the amino acid sequence in each frame, and then the amino acid sequence is compared with the amino acid sequence of the well-known periostin variant (ΔbΔe), thus confirming whether or not the obtained cDNA is DNA encoding the periostin variant (ΔbΔe). By using the obtained DNA encoding the periostin variant (ΔbΔe), an appropriate length DNA fragment is prepared if necessary.

A recombinant expression vector of the protein is constructed by inserting the DNA fragment or the full-length cDNA downstream of a promoter in an expression vector. Specifically, an expression vector, in which cDNA encoding a polypeptide consisting of the amino acid sequence of SEQ ID NO: 2 (human sequence), SEQ ID NO: 5 (mouse sequence), or SEQ ID NO: 8 (rat sequence), or a full-length cDNA including nucleotide sequences of SEQ ID NO: 1 (human sequence), SEQ ID NO: 4 (mouse sequence), or SEQ ID NO: 7 (rat sequence) has been inserted, is created.

The recombinant expression vector is introduced into a host cell corresponding to the expression vector. As the host cell, any host cells can be used as long as they can express the targeted DNA, and examples of the host cells may include bacteria belonging to genus Escherichia, genus Serratia, genus Corynebacterium, genus Brevibacterium, genus Pseudomonas, genus Bacillus, genus Microbacterium, and the like, yeast belonging to genus Kluyveromyces, genus Saccharomyces, genus Shizosaccharomyces, genus Trichosporon, and genus Schwanniomyces, or animal cells, insect cells, and the like.

As the expression vector, a vector that is capable of autonomous replication in a host cell or being incorporated into a chromosome, and that contains a promoter in a position in which the DNA can be transcribed is used.

When a bacteria is used as a host cell, it is preferable that the DNA recombinant expression vector is capable of autonomous replication in the bacteria, and is a recombinant expression vector composed of a promoter, a ribosome binding sequence, DNA of the polypeptide and a transcription termination sequence. The vector may include a gene for controlling a promoter.

Examples of the expression vector may include pBTrp2, pBTac1, and pBTac2 (all of them are manufactured by Boehringer Mannheim), pKK233-2 (manufactured by Amersham Pharmacia Biotech), pSE280 (manufactured by Invitrogen), pGEMEX-1 (manufactured by Promega), pQE-8 (manufactured by QIAGEN), pKYP10 [Japanese Patent Application Laid-Open No. 58-110600], pKYP200 [Agricultural Biological Chemistry, 48, 669 (1984)], pLSA1 [Agric. Biol. Chem., 53, 277 (1989)], pGEL1 [Proc. Natl. Acad. Sci. USA, 82, 4306 (1985)], pBluescript II SK(−) (manufactured by Stratagene), pGEX (manufactured by Amersham Pharmacia Biotech), pET-3 (manufactured by Novagen), pTerm2 (USP4686191, USP4939094, and USP5160735), pSupex, pUB110, pTP5, pC194, pEG400 [J. Bacteriol., 172, 2392 (1990)], and the like.

As the expression vector, it is preferable to use a vector in which a distance between Shine-Dalgarno sequence as a ribosome binding sequence and a start codon is adjusted to an appropriate distance (for example, 6 to 18 nucleotides).

As a promoter, any promoters can be used as long as they function in a host cell. Examples of the promoters may include promoters derived from Escherichia coli such as a trp promoter (Ptrp), a lac promoter (Plac), a P_{L} promoter, a P_{R} promoter, and a T7 promoter, phage, and the like, a SPO1 promoter, SPO2 promoter, a penP promoter, and the like. In addition, an artificially designed and modified promoter such as a promoter in which two Ptrps are connected in series (Ptrpx2), a tac promoter, a letI promoter [Gene, 44, 29 (1986)], and a lacT7 promoter can be used.

A nucleotide sequence of DNA of a portion encoding a periostin variant (ΔbΔe) is substituted to be a codon suitable for an expression of a host cell, thereby enabling the productivity of the target polypeptide or protein to be improved. Although a transcription termination sequence is not necessarily required for expression of DNA encoding the above-mentioned polypeptide or protein, it is preferable to dispose a transcription termination sequence immediately downstream of a structural gene.

Examples of the host cell may include a microorganism such as genus Escherichia, genus Serratia, genus Corynebacterium, genus Brevibacterium, genus Pseudomonas, genus Bacillus, genus Microbacterium, and the like, and examples may include Escherichia coli XL1-Blue, Escherichia coli XL2-Blue, Escherichia coli DH1, Escherichia coli MC1000, Escherichia coli KY3276, Escherichia coli W1485, Escherichia coli JM109, Escherichia coli HB101, Escherichia coli No. 49, Escherichia coli W3110, Escherichia coli NY49, Bacillus subtilis, Bacillus amyloliquefaciens, Brevibacterium ammoniagenes, Brevibacterium immariophilum ATCC14068, Brevibacterium saccharolyticum ATCC14066, Corynebacterium glutamicum ATCC13032, Corynebacterium glutamicum ATCC14067, Corynebacterium glutamicum ATCC13869, Corynebacterium acetoacidophilum ATCC13870, Microbacterium ammoniaphilum ATCC15354, Pseudomonas sp. D-0110, and the like.

As the method for introducing a recombinant expression vector, any methods can be used as long as they are methods of introducing a DNA into the above-mentioned host cell, and examples may include a method using a calcium ion [Proc. Natl. Acad. Sci. USA, 69, 2110 (1972)], a protoplast method [Japanese Patent Application Laid-Open No. 63-248394], a method described in Gene, 17, 107 (1982) and Molecular & General Genetics, 168, 111 (1979), and the like.

When yeast is used as a host, examples of the expression vector may include YEp13 (ATCC:37115), YEp24 (ATCC:37051), YCp50 (ATCC:37419), pHS19, pHS15 and the like.

As the promoter, any promoters can be used as long as they function in yeast, and examples may include a PHO5 promoter, a PGK promoter, a GAP promoter, an ADH promoter, a gal 1 promoter, a gal 10 promoter, a heat-shock protein promoter, an MFα1 promoter, a CUP 1 promoter, and the like.

Examples of the host cell may include Saccharomyces cerevisiae, Schizosaccharomyces pombe, Kluyveromyces lactis, Trichosporon pullulans, Schwanniomyces alluvius, and the like.

As the method of introducing a recombinant vector, any methods can be used as long as they are methods of introducing a DNA into yeast, and examples may include an electroporation method [Methods. in Enzymol., 194, 182 (1990), a spheroplast method [Proc. Natl. Acad. Sci., USA, 75, 1929 (1978)], a lithium acetate method [J. Bacteriol., 153, 163 (1983)], a method described in Proc. Natl. Acad. Sci. USA, 75, 1929 (1978), and the like.

When an animal cell is used as a host cell, as an expression vector, for example, pcDNA1.1 (manufactured by Invitrogen), pCDM8 (manufactured by Invitrogen), pAGE107 [Japanese Patent Application Laid-Open No. 3-22979; Cytotechnology, 3, 133 (1990)], pAS3-3 (Japanese Patent Application Laid-Open No. 2-227075), pcDNA1.1/Amp (manufactured by Invitrogen), pREP4 (manufactured by Invitrogen), pAGE103 [J. Biochem., 101, 1307 (1987)], pAGE210, and the like, can be used.

As the promoter, any promoters may be used as long as they function in an animal cell, and examples may include a promoter of IE (immediate early) gene of human cytomegalovirus (HCMV), an early promoter of SV40, a promoter of a retrovirus, a metallothionein promoter, a heat-shock protein promoter, an SRα promoter, and the like. Furthermore, an enhancer of a human CMV IE gene may be used together with a promoter.

Examples of the host cell may include a Namalwa cell as a human cell, a COS cell as a monkey cell, a CHO cell as Chinese hamster cell, HBT5637 [Japanese Patent Application Laid-Open No. 63-299], and the like.

As the method of introducing a recombinant vector, any methods can be used as long as they are methods of introducing a DNA into an animal cell, and examples may include an electroporation method [Cytotechnology, 3, 133 (1990)], a calcium phosphate method (Japanese Patent Application Laid-Open No. 2-227075), a lipofection method [Proc. Natl. Acad. Sci., USA, 84, 7413 (1987), Virology, 52, 456 (1973)], and the like.

When an insect cell is used as the host cell, for examples, a polypeptide or protein can be expressed by the method described in Baculovirus Expression Vectors, A Laboratory Manual), Current Protocols in Molecular Biology supplement 1-38 (1987-1997), Bio/Technology, 6, 47 (1988), and the like.

That is to say, a polypeptide or protein can be expressed by co-introducing a recombinant gene-introduction vector and a baculovirus into an insect cell to obtain a recombinant virus in an insect cell culture medium, and then infecting the insect cell with a recombinant virus. Examples of the vector for gene introduction may include pVL1392, pVL1393, and pBlueBacIII (all of them are manufactured by Invitrogen). Examples of the baculovirus may include Autographa California nuclear polyhedrosis virus, which infects a cabbage army worm family insect, and the like.

As the insect cell, Sf9 as an ovarian cell of Spodoptera frugiperda, Sf21 [Baculovirus Expression Vectors, A Laboratory Manual, W.H. Freeman and Company, New York, (1992)], High 5 as an ovarian cell of Trichoplusia ni (manufactured by Invitrogen) can be used.

As a method of co-introducing the above-mentioned recombinant gene-introduction vector and the above-mentioned baculovirus into an insect cell for preparing the recombinant virus, for example, a calcium phosphate method [Japanese Patent Application Laid-Open No. 2-227075], a lipofection method [Proc. Natl. Acad. Sci., USA, 84, 7413 (1987)], and the like, can be used.

As the expression method of a gene, in addition to the direct expression, secretory production, fusion protein expression, and the like, can be carried out according to the method described in Molecular Cloning, 2nd Edition.

When expression is carried out in yeast, an animal cell, or an insect cell, a polypeptide or protein to which sugar or a sugar chain has been added can be obtained.

When a transformant harboring a recombinant DNA in which DNA of the protein has been incorporated is cultured in a medium, a target polypeptide or a target protein is produced and accumulated in the cultured product, and the polypeptide or protein is collected from the cultured product. Thus, the target polypeptide or the target protein can be produced. A method for culturing a transformant for producing a periostin variant (ΔbΔe) in a medium can be carried out by the usual method used for culturing a host cell.

When the above-mentioned transformant uses a prokaryote such as Escherichia coli or an eukaryote such as yeast as a host cell, a medium for culturing such a transformant may be a natural medium and a synthetic medium as long as it is a medium which contains a carbon source, a nitrogen source, inorganic substance, and the like that can be utilized by the host cell, and in which the transformant can be cultured efficiently.

As the carbon source, any carbon sources may be used as long as they can be utilized by each host cell, and carbohydrates such as glucose, fructose, sucrose, molasses containing them, starch and starch hydrolysate, organic acids such as acetic acid and propionic acid, and alcohols such as ethanol and propanol can be used.

As the nitrogen source, ammonia, ammonium salts of various inorganic acids and organic acids such as ammonium chloride, ammonium sulfate, ammonium acetate, and ammonium phosphate, other nitrogen-containing substances, as well as peptone, meat extract, yeast extract, corn steep liquor, casein hydrolysate, soybean cake and soybean cake hydrolysate, various fermenting bacterial cells and digestion products thereof, and the like can be used.

As the inorganic substance, potassium dihydrogen phosphate, dipotassium hydrogen phosphate, magnesium phosphate, magnesium sulfate, sodium chloride, ferrous sulfate, manganese sulfate, copper sulfate, calcium carbonate, and the like, can be used.

Culturing is carried out under the aerobic condition, such as shaking culture and deep aeration spinner culture. A culturing temperature is preferably 15 to 40° C., and a culturing time is usually 16 hours to 7 days. During culturing, pH is retained at 3.0 to 9.0. The pH is adjusted using an inorganic or organic acid, an alkali solution, urea, calcium carbonate, ammonia, or the like. If necessary, during culturing, an antibiotic such as ampicillin and tetracycline may be added to a medium.

When a transformant with an expression vector using an inductive promoter as a promoter is cultured, an inducer may be added to a medium, if necessary. For example, when a transformant using an expression vector using a lac promoter is cultured, isopropyl-β-D-thiogalactopyranoside (IPTG) or the like may be added to a medium. When a transformant using an expression vector using a trp promoter is cultured, indoleacrylic acid (IAA) or the like may be added to a medium.

As a medium for culturing a transformant obtained by using an animal cell as a host cell, an RPMI1640 [The Journal of the American Medical Association, 199, 519 (1967)], an Eagle's MEM medium [Science, 122, 501 (1952)], a Dulbecco's modified MEM medium [Virology, 8, 396 (1959)], a 199 medium [Proceeding of the Society for the Biological Medicine, 73, 1 (1950)], or a medium obtained by adding a bovine fetal serum or the like to these media, which are generally used, can be used.

Culturing is usually carried out for one to seven days under the conditions of pH 6 to 8, at 30 to 40° C., and in the presence of 5% CO₂. In addition, during culturing, an antibiotic such as kanamycin and penicillin may be added to a medium, if necessary.

As a medium for culturing a transformant obtained by using an insect cell as a host cell, a TNM-FH medium (manufactured by Pharmingen), a Sf-900II SFM medium (manufactured by Life Technologies), ExCell400, ExCell405 (manufactured by JRH Biosciences), Grace's Insect Medium [Grace, T.C.C., Nature, 195, 788 (1962)], and the like, which are generally used, can be used. Culturing is usually carried out for one to five days under the conditions of pH 6 to 7, and at 25 to 30° C. In addition, during culturing, an antibiotic such as gentamicin may be added to a medium, if necessary.

For isolating and purifying the target polypeptide or the target protein from a cultured product of a transformant, usual isolation and purification method of a polypeptide or protein may be carried out. For example, when the polypeptide or protein is produced in a dissolve state in cells, after culturing is completed, the cells are harvested by centrifugation, suspended in an aqueous buffer, and then, a cell-free extract is prepared by disrupting the cells with an ultrasonic disintegrator, a French press, a Manton Gaurin homogenizer, a Dyno mill, or the like. The cell-free extract is centrifuged, and then, a purified preparation of the polypeptide or protein can be obtained from the obtained supernatant by commonly used methods for protein or polypeptide isolation and purification, including techniques such as a solvent extraction method, salting-out with ammonium sulfate or the like, desalting, precipitation with organic solvents, anion-exchange chromatography using resin such as diethylaminoethyl (DEAE)-Sepharose and DIAION HPA-75 (manufactured by Mitsubishi Chemical), cation-exchange chromatography using resin such as S-Sepharose FF (manufactured by Amersham Pharmacia Biotech), hydrophobic chromatography using resin such as butyl Sepharose and phenyl Sepharose, a gel filtration method using molecule sieve, affinity chromatography, a chromatofocusing method, electrophoresis such as isoelectric electrophoresis, and the like. These techniques can be used either alone or in combination.

Furthermore, when a polypeptide or protein is produced as an inclusion body in cells, the cells are harvested, crushed, centrifuged, and then, the inclusion body of the polypeptide or protein is recovered as precipitated fraction. The recovered inclusion bodies of the polypeptide or protein is solubilized with a protein denaturant. The solubilized solution is diluted or dialyzed so as to reduce the concentration of the protein denaturant in the solubilized solution, thereby returning the conformation of the polypeptide or protein to a normal three-dimensional conformation. Thereafter, a purified preparation of the polypeptide or protein is obtained by the same protein isolation and purification method described above.

When a polypeptide or protein, glycosylated form thereof, or the like, is secreted extracellularly, the polypeptide or protein, the glycosylated form thereof, or the like, can be recovered from culture medium. That is to say, a culture medium is collected from the culture product by techniques such as centrifugation, and a purified preparation can be obtained from the culture medium by the isolation and purification method described above.

Examples of the thus obtained polypeptide or protein can include a polypeptide and the like having amino acid sequences of SEQ ID NO: 2, SEQ ID NO: 3, SEQ ID NO: 5, SEQ ID NO: 6, SEQ ID NO: 8, and SEQ ID NO: 9.

Furthermore, the polypeptide or protein can be produced by a chemical synthesizing method such as the Fmoc method (fluorenylmethyloxycarbonyl method) and tBoc method (t-butyloxycarbonyl method). Alternatively, the polypeptide or protein can be synthesized by using a peptide synthesizer commercially available from Advanced ChemTech (USA), Perkin-Elmer, Amersham Pharmacia Biotech, Protein Technology Instrument (USA), Synthecell-Vega (USA), PerSeptive (USA), Shimadzu Corporation, and the like.

2. Production of Antibody Specifically Recognizing Periostin Variant (ΔbΔe) Protein

By using a partial fragment purified preparation of the polypeptide or protein obtained in the above-mentioned 1, or the amino acid sequences of SEQ ID NO: 2, SEQ ID NO: 3, SEQ ID NO: 5, SEQ ID NO: 6, SEQ ID NO: 8, and SEQ ID NO: 9, it is possible to produce an antibody such as a polyclonal antibody and a monoclonal antibody for recognizing the periostin variant (ΔbΔe) protein.

### (1) Production of Polyclonal Antibody

A polyclonal antibody can be produced by using a purified preparation of a periostin variant (ΔbΔe) protein or a partial fragment polypeptide of the protein, or a peptide having a part of the amino acid sequence of the protein as an antigen, and administering it to an animal. In particular, it is preferable that a region that is different from the full-length periostin sequence is used as an antigen, but a common sequence may be used as an antigen as long as it can specifically recognize a periostin variant (ΔbΔe) protein. As an animal to be administered, rabbit, goat, rat, mouse, hamster, and the like, can be used.

A dose of the antigen is preferably 50 to 100 μg per animal. When a peptide is used, it is desirable to use an antigen covalently bound to a carrier protein such as keyhole limpet haemocyanin, cow thyroglobulin, or the like. A peptide which is to be an antigen can be synthesized by using a peptide synthesizer.

Administration of the antigen is performed 3 to 10 times every 1 to 2 weeks after first administration. After each administration, blood is collected from an eyeground venous plexus on Day 3 to 7, and a reaction of the serum with an antigen used in immunization can be confirmed by enzyme-linked immunosorbent assay and the like [Enzyme-linked Immunosorbent Assay (ELISA method): published by Igaku-Shoin Ltd. (1976), Antibodies: A Laboratory Manual, Cold Spring Harbor Laboratory (1988)].

To the antigen used for immunization, a serum is obtained from non-human mammalian that is immunized to such an extent that a sufficient antibody titer is obtained, and the serum is separated and purified so as to obtain a polyclonal antibody. A separation and purification method can be performed by centrifugation, salting out with 40 to 50% saturation ammonium sulfate, caprylic acid precipitation [Antibodies, A Laboratory manual, Cold Spring Harbor Laboratory, (1988)], or various chromatographies using a DEAE-Sepharose column, an anion exchange column, a protein A- or G-column or a gel filtration column, alone or in combination thereof. In order to increase the specificity of the produced antibody, it is preferable that affinity purification with a periostin variant (ΔbΔe) column, or absorption and purification with a full-length periostin or other periostin variants is carried out.

### (2) Production of Monoclonal Antibody

### (a) Preparation of Antibody Producing Cell

As an immunogen, a protein or peptide that can be used for producing the polyclonal antibody can be used. With respect to the protein or the partial fragment polypeptide used in the immunization, the serum of a mouse or a rat exhibiting a sufficient antibody titer is used as a supplying source of an antibody producing cell. On Day 3 to 7 after an antigen substance is finally administered to the mouse or rat exhibiting the sufficient antibody titer, the spleen is extracted.

The spleen is finely cut in an MEM medium (manufactured by Nissui Pharmaceutical Co., LTD), loosened with a forceps, and centrifuged at 1,200 rpm for 5 minutes, and the supernatant is discarded. The spleen cell in the obtained precipitation fraction is treated with a Tris-ammonium chloride buffer (pH 7.65) for 1 to 2 minutes to remove erythrocyte, which is then washed with an MEM medium three times. The resulting spleen cell is used as an antibody-producing cell.

### (b) Preparation of Myeloma Cell

As the myeloma cell, a cell line obtained from a mouse or a rat is used. For example, an 8-azaguanine-registant mouse (BALE/c-derived) myeloma cell, a P3-X63Ag8-U1 strain (hereinafter, abbreviated as P3-U1) [Curr. Topics. Microbiol. Immunol., 81, 1 (1978), Europ. J. Immunol., 6, 511 (1976)], SP2/0-Ag14 (SP-2) [Nature, 276, 269 (1978)], P3-X63-Ag8653 (653) [J. Immunol., 123, 1548 (1979)], P3-X63-Ag8 (X63) [Nature, 256, 495 (1975)] and the like can be used. These cell strains are subcultured in an 8-azaguanine medium [a medium obtained by adding glutamine (1.5 mmol/L), 2-mercaptoethanol (5×10⁻⁵ mol/L), gentamycin (10 μg/ml) and fetal calf serum (FCS) (manufactured by CSL, 10%) to RPMI-1640 medium (hereinafter, referred to as “normal medium”), and further adding an 8-azaguanine (15 μg/ml) thereto], and cultured in the normal medium 3 to 4 days before cell fusion. In cell fusion, 2×10⁷ or more of the cells are used.

### (c) Production of Hybridoma

The antibody-producing cell prepared in (a) and the myeloma cell prepared in (b) are well washed with an MEM medium or PBS (1.83 g of disodium phosphate, 0.21 g of monopotassium phosphate, 7.65 g of saline, and 1 litter of distilled water, pH7.2), and they are mixed so that the number of cells satisfies the ratio of antibody producing cells:myeloma cells=5 to 10:1. The mixture is centrifuged at 1,200 rpm for 5 minutes and the supernatant is discarded.

A cell group of the resulting precipitation fraction is loosened well, and to the cell group, 0.2 to 1 ml of a mixture solution of 2 g polyethylene glycol-1000 (peg-1000), 2 ml of MEM, and 0.7 ml of dimethyl sulfoxide (DMSO) per 10⁸ antibody-producing cells are added while stirring at 37° C. and, further, 1 to 2 ml of an MEM medium is added a few times every 1 to 2 minutes.

After addition, the MEM medium is added so that the total amount is adjusted to 50 mL. This solution is centrifuged at 900 rpm for 5 minutes and the supernatant is discarded. The cells of the obtained precipitation fraction are slowly loosened, and slowly suspended in 100 mL of HAT medium [medium obtained by adding hypoxanthine (10⁻⁴ mol/L), thymidine (1.5×10⁻⁵ mol/L) and aminopterin (4×10⁻⁷ mol/L) to a normal medium] by suction and blow-out by using a measuring pipette.

Each 100 μl of the suspension is dispensed in each well of a 96-well culture plate, and this is cultured in 5% CO₂ incubator at 37° C. for 7 to 14 days.

After culture, a part of the culture medium is taken out, and a hybridoma specifically reacting to full-length periostin variant (ΔbΔe) or a partial fragment polypeptide is selected by an enzyme immunoassay described in, for example, Antibodies [Antibodies, A Laboratory manual, Cold Spring Harbor Laboratory, Chapter 14 (1988)].

Specific examples of the enzyme immunoassay may include the following methods. Full-length or partial fragment polypeptide of periostin variant (ΔbΔe) used as an antigen in immunization is coated on an appropriate plate, which is reacted with a hybridoma culture medium or a purified antibody obtained in the below-mentioned (d) as a first antibody, and further reacted with an anti-rat or anti-mouse immunoglobulin antibody, which is labeled with biotin, enzyme, chemical luminescence substance, or a radioactive compound, and the like, as a second antibody and then reaction is carried out corresponding to the labeled substances. Then, hybridoma specifically reacting to the periostin variant (ΔbΔe) is selected as a hybridoma producing a monoclonal antibody of the present invention. Furthermore, it is preferable that reactivity with respect to the full-length periostin or other periostin variants is examined and then hybridoma that is not reacted with the protein is selected.

By using the hybridoma, cloning is repeated twice by the limiting dilution method [the first cloning uses an HT medium (a medium obtained by removing aminopterin from the HAT medium), and the second cloning uses a normal medium]. Then, a hybridoma exhibiting a stably strong antibody titer is selected as a hybridoma strain for producing a monoclonal antibody to be used in the present invention.

### (d) Preparation of Monoclonal Antibody

To 8-10 week-old mice or nude mice subjected to pristane treatment [0.5 mL of 2,6,10,14-tetramethylpentadecane (Pristane) is administered intraperitoneally to breed for two weeks], 5−20×10⁶ cells/mouse of the monoclonal antibody producing hybridoma cells obtained in (c) are intraperitoneally injected. In 10-21 days, the hybridoma has ascites carcinoma.

The ascites is collected from a mouse with ascites carcinoma, and centrifuged at 3,000 rpm for 5 minutes so as to remove the solid parts. From the obtained medium, a monoclonal antibody can be purified and obtained by the same method as a method used in the case of a polyclonal antibody.

The determination of subclass of the antibody is carried out by using a mouse monoclonal antibody typing kit or a rat monoclonal antibody typing kit. The amount of polypeptide is calculated by a Lowry method or by the absorbance at 280 nm.

3. Detection Method of mRNA of Periostin Variant (ΔbΔe) Gene by Using DNA Encoding Periostin Variant (ΔbΔe)

Examples of the DNA to be used in the detection method may include a DNA consisting of the nucleotide sequences of SEQ ID NO: 1, SEQ ID NO: 4, and SEQ ID NO: 7, a DNA fragment obtained therefrom, or the like. Hereinafter, these are also referred to as “DNA to be used in the present invention.” Examples of the methods for detecting the expression level of mRNA of the gene and structural change include (1) Northern blotting, (2) in situ hybridization method, (3) quantitative PCR method, (4) differential hybridization method, (5) DNA chip method, (6) RNase protection assay, and the like.

Specimens that can be used for the analysis by the above-mentioned method include biological samples such as heart tissues, blood (collected peripheral blood, collected coronary sinus, and the like), serum thereof, and saliva which are collected from patients with diseases with myocardial necrosis and healthy persons, or mRNA or total RNA collected from a primary cultured cell sample obtained by culturing a cell from the biological samples in an appropriate medium in a test tube (hereinafter, the mRNA and total RNA are referred to as “specimen-derived RNA”). Furthermore, the tissue collected from the biological sample, which is isolated as paraffin or cryostat sections, can be also used.

Northern blotting includes separation by the gel electrophoresis of specimen-derived RNA, followed by transferring the separated RNA onto a supporting material such as a nylon filter, carrying out hybridization using a labeled probe prepared from a DNA to be used in the present invention, washing and detecting the band specifically bound to the gene mRNA. Thus, it is possible to detect the expression level and the structural change of the gene mRNA. In hybridization, incubation is carried out under conditions in which the probe and the gene mRNA in the specimen-derived RNA form a stable hybrid. In order to prevent the false-positive reactions, the hybridization and washing steps are desirably carried out in the highly stringent conditions. Such conditions can be determined by various factors such as temperature, ionic strength, base composition, length of probe, formamide concentration, and the like. These factors are described in, for example, Molecular Cloning, 2nd Ed.

The labeled probe to be used in Northern blotting can be prepared, for example, by incorporating a radioisotope, biotin, a fluorescent group, a chemiluminescent group, or the like, into a DNA to be used in the present invention or an oligonucleotide designed based on the sequence of the DNA by a well-known method (nick-translation, random priming or kinasing). The amount of bound labeled probes reflects an expression level of the gene mRNA. Thus, the expression level of the gene mRNA can be determined by quantifying the amount of the bound labeled probes. Furthermore, the structural change of the gene mRNA can be detected by analyzing the binding site of the labeled probe.

In situ hybridization is a method for detecting the mRNA expression level of the gene by carrying out the steps of hybridization using the above-mentioned labeled probe and paraffin or cryostat sections of tissues obtained from a living body and washing. In order to prevent false-positive reactions during in situ hybridization, it is desirable to carry out the steps of hybridization and washing under highly stringent conditions. The conditions can be determined based on various factors such as temperature, ionic strength, base composition, length of probe, and formamide concentration. These factors are described, for example, in Current Protocols in Molecular Biology.

Methods for detecting mRNA of the gene, such as quantitative PCR, a differential hybridization method, and a DNA-chip method, can be carried out by the method based on synthesizing cDNA from specimen-derived RNA using an oligo dT primer or random primer or reverse transcriptase (hereinafter, the cDNA is referred to as “specimen-derived cDNA”). When the specimen-derived RNA is mRNA, both of the above-mentioned primers can be used, whereas when the specimen-derived RNA is total RNA, the oligo dT primer is used.

In quantitative PCR, DNA fragments derived from mRNA of the gene are amplified by PCR using a specimen-derived cDNA as a template and primers designed based on the nucleotide sequence of a DNA encoding a periostin variant (ΔbΔe). The amount of the amplified DNA fragments reflects an expression level of the mRNA of the gene. Thus, the amount of the mRNA of the gene can be quantified by using a DNA encoding actin, G3PDH (glyceraldehyde 3-phosphate dehydrogenase), or the like as an internal control. Further, the structural change of mRNA of the gene can be detected by separating the amplified DNA fragments by gel electrophoresis. According to this detection method, it is desirable to use primers that are suitable for specific and efficient amplification of the target sequence. Such suitable primers can be designed based on conditions where neither inter-primer nor intra-primer base pairing is formed, and where the primers specifically bind to the target cDNAs at a certain annealing temperature and dissociate from the target cDNAs by denaturation. The quantification of the amplified DNA fragments must be carried out within a PCR reaction in which amplified products are exponentially increased. Such a PCR reaction can be identified by recovering DNA fragments amplified in each reaction and quantitatively analyzing them by gel electrophoresis.

Differential hybridization [Trends in Genetics, 7, 314-317 (1991)] and the DNA chip method [Genome Research, 6, 639-645 (1996)] are methods for detecting change in the expression level of mRNA of the gene by carrying out hybridization and washing on a filter or a base such as slide glass or silicon, on which a DNA to be used in the present invention has been immobilized, using a specimen-derived cDNA as a probe. According to either method, the differences in expression level of mRNA of the gene between control and target specimens can be accurately detected by immobilizing actin, G3PDH, or the like, as an internal control on the filter or base. Alternatively, the expression level of mRNA of the gene can be accurately quantified by synthesizing labeled cDNA based on RNA of control specimen and target specimen-derived RNA by using different labeled dNTPs, and then hybridizing two labeled cDNA probes simultaneously on the filter or base.

The RNase protection assay can be carried out by the following procedure. Firstly, a promoter sequence such as T7 promoter and SP6 promoter is linked to the 3′-terminal of a DNA to be used in the present invention. A labeled antisense RNA is synthesized by in vitro transcription system that uses RNA polymerase and labeled rNTP. The labeled antisense RNA is bound to specimen-derived RNA to hybridize RNA-RNA hybrid. The resulting RNA-RNA hybrid is digested with RNase, and then the RNA fragment protected from digestion is detected as a band after gel electrophoresis. The resulting band is quantified, thereby enabling the expression level of mRNA of the gene to be quantified.

4. Immunological Detection or Quantitation Method Using Antibody Specifically Recognizing Periostin Variant (ΔbΔe) Protein

Immunological methods for detecting and quantifying a periostin variant (ΔbΔe) protein intracellulary or extracellulary expressed by microorganisms, animal cells, insect cells or tissues using an antibody (polyclonal or monoclonal antibody) that specifically recognizes a periostin variant (ΔbΔe) protein include a fluorescent antibody method, an enzyme-linked immunosorbent assay (ELISA), radioimmunoassay (RIA), an immunohistochemistry staining method (ABC method, CSA method, and the like) such as an immunohistological staining method and an immunocytological staining method, a Western blotting method, a dot blotting method, immunoprecipitation, sandwich ELISA, and an immunoagglutination method [Monoclonal Antibody—Experimental Manual, Kodansha Scientific (1987); The second series of lectures on biochemical experiments Vol. 5, Immunobiochemical Experiments, Tokyo Kagaku Dozin (1986)].

Specimens that can be used for the analysis by the above-mentioned methods include biological samples such as heart tissues, blood (collected peripheral blood, collected coronary sinus, and the like), serum thereof, and saliva which are collected from patients with diseases with myocardial necrosis and healthy persons, or protein collected from a primary cultured cell sample obtained by culturing a cell from the biological sample in an appropriate medium in a test tube. Furthermore, the tissue, which is collected from the biological sample and isolated as paraffin or cryostat sections, can be used.

The fluorescent antibody method includes reacting an antibody to be used in the present invention with the above-mentioned samples or the extract in order to examine the presence or absence of periostin variant (ΔbΔe) protein, and further reacting thereto an anti-mouse IgG antibody or a fragment thereof labeled with a fluorescent substance such as fluorescein isothiocyanate (FITC), and measuring the fluorescent dye in flow cytometer.

The enzyme-linked immunosorbent assay (ELISA) includes reacting an antibody to be used in the present invention with the above-mentioned samples or the extract in order to examine the presence or absence of periostin variant (ΔbΔe) protein, and further reacting thereto an anti-mouse IgG antibody or a labeled fragment thereof labeled with an enzyme such as peroxidase and biotin, and measuring the color dye by using an absorption spectrophotometer.

The radioimmunoassay (RIA) includes reacting an antibody to be used in the present invention with the above-mentioned samples or the extract in order to examine the presence or absence of periostin variant (ΔbΔe) protein, further reacting thereto an anti-mouse IgG antibody or a fragment thereof labeled with a radioisotope, and then measuring the radioactivity with a scintillation counter, and the like.

The immunohistochemistry staining method such as an immunohistological staining method and an immunocytological staining method includes reacting an antibody to be used in the present invention with the above-mentioned samples or the extract in order to examine the presence or absence of periostin variant (ΔbΔe) protein, further reacting thereto an anti-mouse IgG antibody or a fragment thereof labeled with a fluorescent material such as FITC or an enzyme such as peroxidase and biotin, and observing the label under a microscope.

The Western blotting method includes fractionating the above-mentioned samples or the extract by SDS-polyacrylamide gel electrophoresis [Antibodies-A Laboratory Manual, Cold Spring Harbor Laboratory (1988)], blotting the protein from the gel onto a PVDF membrane or nitrocellulose membrane in order to examine the presence or absence of periostin variant (ΔbΔe) protein, reacting the antibody to be used in the preset invention with the membrane, further reacting thereto an anti-mouse IgG antibody or a fragment thereof labeled with a fluorescent substance such as FITC or an enzyme such as peroxidase and biotin, and then confirming thereof.

The dot blotting method includes blotting the above-mentioned samples or the extract onto a nitrocellulose membrane in order to examine the presence or absence of periostin variant (ΔbΔe) protein, reacting an antibody specifically recognizing periostin variant (ΔbΔe) protein with the membrane, further reacting thereto an anti-mouse IgG antibody or a fragment thereof labeled with a fluorescent substance such as FITC or an enzyme such as peroxidase and biotin, and then confirming thereof.

The immunoprecipitation includes reacting the above-mentioned samples or the extract with an antibody specifically recognizing periostin variant (ΔbΔe) protein in order to examine the presence or absence of periostin variant (ΔbΔe) protein, adding thereto a carrier capable of specifically binding to immunoglobulin, such as protein G-Sepharose and precipitating the antigen-antibody complex.

The sandwich ELISA includes: previously immobilizing one of two antibodies, which specifically recognize a periostin variant (ΔbΔe) protein and which respectively have two different antigen-recognition sites, on a plate, and labeling the other antibody with a fluorescent substance such as FITC or an enzyme such as peroxidase and biotin; reacting the above-mentioned samples or the extract with the antibody immobilized plate in order to examine the presence or absence of periostin variant (ΔbΔe) protein; and reacting the labeled antibody thereto and detecting the labeled substance bound thereto.

The immunoagglutination method includes immobilizing two antibodies (or one antibody), which specifically recognize a periostin variant (ΔbΔe) protein and which respectively have two different antigen-recognition sites, to latex particles or liposome, and reacting thereto with the above-mentioned sample or the extract in order to examine the presence or absence of a periostin variant (ΔbΔe) protein, and detecting the turbidity of agglutinated particles by absorbance and the like.

5. Mutation Identification Method of DNA Encoding Periostin Variant (ΔbΔe)

Examples of DNAs to be used in the method may include DNAs having nucleotide sequences of SEQ ID NO: 1, SEQ ID NO: 4, and SEQ ID NO: 7, or a DNA fragments thereof.

The most apparent test for evaluating the presence or absence of a causative mutation of patients with diseases with myocardial necrosis, which is located within the locus of a periostin variant (ΔbΔe) gene, is a direct comparison of the genes between a control group and the patients with diseases with myocardial necrosis.

Specifically, human biological samples, such as heart tissue, blood (collected peripheral blood, collected coronary sinus, and the like), serum thereof, and saliva are collected from patients with diseases with myocardial necrosis and healthy persons. Alternatively, samples are collected from primary culture cells established from the biological samples. DNAs are extracted from the biological samples or the primary culture cell-derived samples (hereinafter the DNA is referred to as “specimen-derived DNA”). The specimen-derived DNA can be used directly, or a DNA encoding a periostin variant (ΔbΔe) amplified by using primers designed based on the nucleotide sequences of DNA having nucleotide sequences of SEQ ID NO: 1, SEQ ID NO: 4, and SEQ ID NO: 7 can also be used as the sample DNA. Alternatively, DNA fragments amplified by PCR using specimen-derived cDNA as a template and using primers designed based on the nucleotide sequences of DNA having nucleotide sequences of SEQ ID NO: 1, SEQ ID NO: 4, and SEQ ID NO: 7 can be also used as the sample DNA.

As a method for determining the presence or absence of a causative mutation of diseases with myocardial necrosis in a DNA encoding a periostin variant (ΔbΔe), a method for detecting a hetero-duplex formed by the hybridization of a DNA strand containing the wild-type allele to a DNA strand containing the mutant allele can be used.

Methods for detecting a hetero-duplex include (1) a hetero-duplex detection method by polyacrylamide gel electrophoresis [Trends Genet., 7, 5 (1991)]; (2) a hetero-duplex detection method by single-strand conformation polymorphism analysis [Genomics, 16, 325-332 (1993)]; (3) the method of chemical cleavage of mismatches (CCM) [Human Genetics (1996), Tom Strachan and Andrew P. Read, BIOS Scientific Publishers Limited]; (4) the method of enzymatic cleavage of mismatches [Nature Genetics, 9, 103-104 (1996)]; and (5) denaturing gradient gel electrophoresis [Mutat. Res., 288, 103-112 (1993)].

According to the hetero-duplex detection method by polyacrylamide gel electrophoresis, a fragment of DNA encoding a periostin variant (ΔbΔe) is amplified as a fragment shorter than 200 by PCR using a specimen-derived DNA or a specimen-derived cDNA as a template and using primers designed based on the nucleotide sequence of the DNA having nucleotide sequences of SEQ ID NO: 1, SEQ ID NO: 4, and SEQ ID NO: 7, and then the DNA fragment is subjected to polyacrylamide gel electrophoresis to compare the mobility with homo-duplex without mutations. When a hetero-duplex is formed by a mutation in the DNA encoding a periostin variant (ΔbΔe), it shows a lower mobility of the duplex in the gel than that of the homo-duplex without mutations and thus it can be detected as a band that is different from the homo-duplex. For the electrophoresis, commercially available gel [Hydro-link, MDE, manufactured by FMC Corporation, and the like] can be used. Furthermore, insertions, deletions, and substitutions of a single base can be detected in the method using the DNA fragment that is shorter than 200 bp. It is desirable to carry out the hetero-duplex analysis on a single sheet of gel in combination with a single-strand conformation polymorphism analysis as described below.

The single-strand conformation polymorphism analysis (SSCP analysis) is a method of carrying out electrophoresis by using a non-denaturing polyacrylamide gel after denaturation of a DNA encoding a periostin variant (ΔbΔe) that is amplified as a fragment shorter than 200 by PCR using a specimen-derived DNA or specimen-derived cDNA as a template and using primers designed based on the nucleotide sequence of the DNA having nucleotide sequences of SEQ ID NO: 1, SEQ ID NO: 4, and SEQ ID NO: 7. When amplification of DNA is carried out, primers are labeled with a radioisotope or fluorescent dye, or unlabeled amplified products are stained with silver. Thereby, the DNA encoding the amplified periostin variant (ΔbΔe) can be detected as a band. In order to make a difference from the electrophoretic pattern of the wild type, the co-electrophoresis of a control is carried out, and thus, fragments having mutated nucleotide sequences can be detected based on the difference in electrophoretic mobility.

According to the method of chemical cleavage of mismatches (CCM), a DNA fragment encoding a periostin variant (ΔbΔe) is amplified using a specimen-derived DNA or specimen-derived cDNA as a template and primers designed based on the nucleotide sequence of the DNA having nucleotide sequences of SEQ ID NO: 1, SEQ ID NO: 4, and SEQ ID NO: 7. Mutations in the nucleotide sequence can be detected by hybridizing the amplified DNA fragment with a labeled DNA that has been prepared by incorporating a radioisotope or fluorescent dye into the DNA having nucleotide sequences of SEQ ID NO: 1, SEQ ID NO: 4, and SEQ ID NO: 7, and cleaving one of the DNA strands at the mismatched position by osmium-tetroxide treatment. The CCM method is one of the most sensitive detection methods, and is applicable to specimen of kilobase-length.

A mismatch can be cleaved enzymatically by the combined use of RNaseA and, instead of the use of the above-mentioned osmium tetroxide, other enzymes such as T4 phage resolvase or endonuclease VII that are associated with the repair of intracellular mismatches.

According to denaturing gradient gel electrophoresis (DGGE method), a DNA fragment encoding a periostin variant (ΔbΔe) is amplified using a specimen-derived DNA or specimen-derived cDNA as a template, and primers designed based on the nucleotide sequence of the DNA having nucleotide sequences of SEQ ID NO: 1, SEQ ID NO: 4, and SEQ ID NO: 7, and then, the amplified DNA fragment is subjected to electrophoresis on a gel with a concentration gradient or a temperature gradient of a chemical denaturant. The amplified DNA fragment moves to a position in the gel where the DNA is denatured to single-stranded chains, and the DNA no longer moves after denaturation. Mutations can be detected based on the differences in the mobility of the amplified DNA in the gel, depending on the presence or absence of mutations in the DNA encoding a periostin variant (ΔbΔe). By adding a poly(G:C) end to primers, PCR to be used can improve the detection sensitivity.

An alternative method for detecting causative genes of diseases with myocardial necrosis includes the protein truncation test (PTT method) [Genomics, 20, 1-4 (1994)]. According to the test, a frame-shift mutation, splice-site mutation and nonsense mutation, all of which may result in protein deficiency, can be specifically detected. In the PTT method, a primer in which a T7 promoter sequence and an eukaryotic translation initiation sequence are linked to the 5′ terminal of the DNA having the nucleotide sequences of SEQ ID NO: 1, SEQ ID NO: 4, and SEQ ID NO: 7 is designed, and by using the primer, cDNA encoding a periostin variant (ΔbΔe) is prepared from a specimen-derived RNA by a reverse transcription-PCR (RT-PCR) method. Proteins can be produced by in vitro transcription and translation using the cDNA. Then, the protein is subjected to polyacrylamide electrophoresis on a gel. When the position of the protein after electrophoresis corresponds to that of a full-length protein, no mutation resulting in protein deficiency exists in the gene. On the other hand, when the protein has deficiency, such a protein migrates to a position which corresponds to that of a shorter protein than the full-length protein. Thus, the degree of deficiency can be detected from the position.

In order to determine the nucleotide sequences of specimen-derived DNA and specimen-derived cDNA, primers designed based on the DNA having nucleotide sequences of SEQ ID NO: 1, SEQ ID NO: 4, and SEQ ID NO: 7 can be used. By analyzing the determined nucleotide sequence, the presence or absence of causative mutations of diseases with myocardial necrosis in the specimen-derived DNA or specimen-derived cDNA can be determined.

Mutations located outside the coding region of a periostin variant (ΔbΔe) gene may be detected by analyzing non-coding regions such as regions in the vicinity of the gene, introns thereof, and regulatory sequence thereof. Diseases with myocardial necrosis caused by mutations in the non-coding regions can be detected by the same method as described above.

The gene, which has been suggested to have a mutation in the non-coding region by the method as described above, can be cloned using, as a hybridization probe, a DNA having nucleotide sequences of SEQ ID NO: 1, SEQ ID NO: 4, and SEQ ID NO: 7. The mutation in the non-coding region can be found according to any of the above-mentioned methods.

An identified mutation can be analyzed according to the statistical method described in Handbook of Human Genetics Linkage (The John Hopkins University Press, Baltimore (1994)) so as to identify the mutation as SNPs (Single nucleotide polymorphism) linked to diseases with myocardial necrosis. Furthermore, a causative gene of diseases with myocardial necrosis can be identified by obtaining DNAs from a family having history of a disease with myocardial necrosis according to the method described above and detecting mutations therein.

6. Diagnostic method of Disease with Myocardial Necrosis Using DNA Encoding Periostin Variant (ΔbΔe)

DNAs to be used in the above-mentioned method include, for example, DNAs having the nucleotide sequences of SEQ ID NO: 1, SEQ ID NO: 4, and SEQ ID NO: 7, and DNA fragments thereof.

The cause of diseases with myocardial necrosis can be identified by detecting mutations in a gene in any human tissues. For example, when a mutation exists in the germ line, an individual who has inherited the mutation may have a tendency to have a disease with myocardial necrosis. The mutation can be detected by testing a DNA extracted from any of tissues of the individual. For example, the risk of diseases with myocardial necrosis can be tested by extracting a DNA from cells of collected human blood and detecting gene mutations using the DNA. Alternatively, the risk of diseases with myocardial necrosis before birth can be tested by collecting amniotic cells, extracting DNA from the cells and detecting gene mutations using the DNA.

Furthermore, the type of diseases with myocardial necrosis can be tested by obtaining a DNA from a living tissue from lesions of a patient who has developed diseases with myocardial necrosis and detecting mutations in genes. This may be useful for selecting drugs to be administered. The DNA of the living tissue can be obtained by isolating a tissue of the lesion liberated from the peripheral normal tissues, treating it with trypsin or the like, culturing the resultant cells in an appropriate culture medium, and extracting a chromosomal DNA and mRNA from the cultured cells.

Furthermore, by extracting biological samples themselves such as heart tissue of a myocardial necrosis site, blood (collected peripheral blood, collected coronary sinus, and the like), serum thereof, urine, feces, and saliva, which are collected from patients with the onset of diseases with myocardial necrosis, or a DNA or mRNA from the cell obtained from the biological samples, and detecting the expression level of gene, the degree or state of the disease with myocardial necrosis is tested, and the state can be monitored by testing the state at the appropriate times.

Specifically, when the expression level of the gene is higher than that of healthy persons with a substantial difference, a disease with myocardial necrosis is defined to be onset. Furthermore, in the monitoring of the disease with myocardial necrosis such as myocardial infarction, an extremely high expression level of the gene shows the dangerous state such as cardiac rupture while a decrease thereof shows the stable phase. Thus, an appropriate treatment policy can be determined.

Hereinafter, a DNA obtained from human specimen by any of the above-mentioned methods for the purpose of testing is referred to as “testing specimen-derived DNA.” Furthermore, a cDNA synthesized from an RNA which is obtained from human specimen by any of the above-mentioned methods for the purpose of testing is referred to as “testing specimen-derived cDNA.”

By using a DNA having nucleotide sequences of SEQ ID NO: 1, SEQ ID NO: 4, and SEQ ID NO: 7, a testing specimen-derived DNA and a testing specimen-derived cDNA, by a method according to the method for detecting a mutation of a DNA encoding a periostin variant (ΔbΔe) in the above-mentioned 5, testing of diseases with myocardial necrosis can be carried out. Furthermore, methods for testing whether or not a testing specimen-derived DNA or a testing specimen-derived cDNA has a mutation that has found to be a cause of diseases with myocardial necrosis by the method mentioned in 5 include: (1) detection of restriction enzyme sites; (2) a method using an allele-specific oligonucleotide probe (ASO: allele specific oligonucleotide hybridization); (3) PCR using allele-specific oligonucleotide (ARMS: amplification refractory mutation system); (4) oligonucleotide ligation assay (OLA); (5) a PCR-PHFA method (PCR-preferential homoduplex formation assay); and (6) a method using an oligo DNA array [Protein, Nucleic Acid and Enzyme, 43, 2004-2011 (1998)].

Hereinafter, the (1) to (6) are described. The detection of restriction enzyme sites can be carried out by the following method. When a restriction enzyme site is lost or generated due to a single base alteration, mutation can be simply detected by amplifying the test specimen-derived DNA or the test specimen-derived cDNA by PCR using primers designed based on the nucleotide sequence of a DNA having nucleotide sequences of SEQ ID NO: 1, SEQ ID NO: 4, and SEQ ID NO: 7, digesting with restriction-enzyme, and comparing the resultant restriction fragments of the DNA with those of a normal person. Furthermore, a method for testing the change in the base may include a method for detecting the mutations by reverse-dot blotting so as to carry out hybridization by designing oligonucleotide probes by combining information of the nucleotide sequence of the DNA having the nucleotide sequences of SEQ ID NO: 1, SEQ ID NO: 4, and SEQ ID NO: 7 as well as the information of mutation identified by the above-mentioned method 5, and binding the oligonucleotide probes to a filter.

The method using allele-specific oligonucleotide probes (ASO) uses a characteristic that a short synthetic DNA probe hybridizes to only a fully-matched nucleotide sequence, thus enabling single-nucleotide mutations to be detected readily. Specifically, this method can be carried out by reverse-dot blotting in which the oligonucleotide designed based on the nucleotide sequence of a DNA having nucleotide sequences of SEQ ID NO: 1, SEQ ID NO: 4, and SEQ ID NO: 7 and the mutation of the base identified in the above-mentioned 5 is bound to a filter, and hybridization is carried out using as a probe an amplified DNA fragment obtained by PCR using a testing specimen-derived DNA or a testing specimen-derived cDNA as a template and primers designed by using the nucleotide sequence of a DNA having nucleotide sequences of SEQ ID NO: 1, SEQ ID NO: 4, and SEQ ID NO: 7 and labeled dNTP.

According to the reverse-dot blotting, oligonucleotides, which have been designed based on the nucleotide sequence of a DNA without mutation corresponding to the DNA to be measured and on the mutations of the separately identified DNA, are synthesized directly on a base such as slide glass and silicon, and then a small amount of a testing specimen-derived DNA or a testing specimen-derived cDNA is reacted to a DNA chip, i.e., a high-density array, thus detecting various mutations more simply. This method is suitable for large scale diagnosis.

Mutations of the base can also be detected by the following oligonucleotide ligation assay (OLA).

Oligonucleotide of about 20 nucleotides having a mutation site of a DNA encoding a periostin variant (ΔbΔe) to be examined at 3′ terminal, and oligonucleotide of about 20 nucleotides following the mutation site are designed and synthesized based on the nucleotide sequence of a DNA having nucleotide sequences of SEQ ID NO: 1, SEQ ID NO: 4, and SEQ ID NO: 7. At this time, two different labels are added. For example, biotin is added to the 5′ terminal of the former oligonucleotide, and digoxigenin is added to the 3′ terminal of the latter oligonucleotide. Next, a DNA encoding a periostin variant (ΔbΔe) is amplified by PCR using a test specimen-derived DNA or a test specimen-derived cDNA as a template and using primers designed based on the nucleotide sequence of the DNA having nucleotide sequences of SEQ ID NO: 1, SEQ ID NO: 4, and SEQ ID NO: 7. Next, the amplified DNA fragment and two of the above-mentioned oligonucleotides are hybridized. After hybridization, the two oligonucleotides are linked to each other with DNA ligase. After linking reaction, a biotin-labeled single-strand DNA obtained by thermally deforming a double-strand DNA is collected as a DNA bound to, for example, avidin. Since the above-mentioned two types of oligonucleotides hybridized to the amplified DNA fragment having a mutation site are linked by the above-mentioned linking reaction, they can be obtained as the DNA having digoxigenin at the 3′ terminal, and the linked DNA can be easily detected. Therefore, the DNA having mutation, which encodes a periostin variant (ΔbΔe), can be detected rapidly and simply. The OLA is a mutation detection method suitable for testing a large number of samples for a short time efficiently because it does not need electrophoresis or centrifugation.

Furthermore, the following PCR-PHFA method allows quantitative and easy detection of a small amount of mutant gene.

The PCR-PHFA method includes: gene amplification (PCR), liquid-phase hybridization with a very high specificity, and ED-PCR (enzymatic detection of PCR product) which detects PCR products by the same procedure as in ELISA. An amplified DNA fragment labeled at both ends is prepared by PCR using a primer set in which one is labeled with DNP (dinitrophenyl) and the other is labeled with biotin, and a DNA having nucleotide sequences of SEQ ID NO: 1, SEQ ID NO: 4, and SEQ ID NO: 7 as a template. Next, a non-labeled primer set having the same nucleotide sequence as that of the labeled primer, a non-labeled amplified DNA that is obtained by amplifying a test specimen-derived DNA or a test specimen-derived cDNA to a template is mixed with the labeled amplified DNA fragment. At this time, the non-labeled amplified DNA fragment is used in an excess amount of 20 to 100 fold of the labeled amplification DNA fragment. The mixture is heat-denatured and cooled under a mild temperature gradient of about 1° C. per 5 min to 10 min, to preferentially form complete complementary strands. The thus reconstituted labeled DNA is trapped and adsorbed on a streptavidin-immobilized well via biotin. An enzyme-labeled anti-DNP antibody is bound thereto via DNP so as to detect by coloring reaction with the enzyme. When no gene having the same sequence as that of the labeled DNA exists in the specimen, original double-stranded labeled DNAs are preferentially reconstituted and as a result the color is developed. On the other hand, when genes having the same nucleotide sequence are present, the amount of reconstituted labeled DNA reduces due to the random replacement of the complementary substitution, resulting in a remarkable decrease of color development. This method enables detection and quantification of known mutations and polymorphic genes.

Furthermore, in order to test the degree or state of diseases with myocardial necrosis, as a method for testing the expression level of genes in the test specimen-derived DNA or test specimen-derived cDNA, the methods described in the above-mentioned 3 are carried out. For example, well-known techniques such as a quantitation PCR method or a micro-array method using a primer that can specifically amplify a periostin variant (ΔbΔe) can be used. Specifically, the method can be carried out by combining SEQ ID NO: 20 and SEQ ID NO: 21.

7. Method for Testing Disease with Myocardial Necrosis Using Antibody Specifically Recognizing Periostin Variant (ΔbΔe) Protein

Identification of the change in the expression level of a periostin variant (ΔbΔe) protein and the structural change of expressed protein in human biological samples is useful in understanding the risk of the onset of a disease with myocardial necrosis, and the cause or degree or state of the already onset deterioration of the cardiac function as well as monitoring the state for use in treatment.

Specifically, when the expression level or degree of structural change of the protein is higher than that of healthy persons with a substantial difference, a disease with myocardial necrosis is defined to be onset. Furthermore, in the monitoring of the disease with myocardial necrosis such as myocardial infarction, an extremely high expression level and degree of structural change of the protein shows the dangerous state such as cardiac rupture while a decrease thereof shows the stable phase. Thus, an appropriate treatment policy can be determined.

The method for detecting and testing the change in the expression level and structure of a periostin variant (ΔbΔe) protein includes a fluorescent antibody method, an enzyme-linked immunosorbent assay (ELISA), radioimmunoassay (RIA), an immunohistochemistry staining method (ABC method, CSA method, and the like) such as an immunohistological staining method and an immunocytological staining method, Western blotting, dot blotting, immunoprecipitation, sandwich ELISA, an immunoagglutination method, and the like, which are described in the above-mentioned 4.

Specimens that can be used for the diagnosis by the above-mentioned method include biological samples themselves such as heart tissues of a lesion site, blood (collected peripheral blood, collected coronary sinus, and the like), serum thereof, urine, feces, and saliva, which are collected from patients, or a cell obtained from the biological samples as well as a cell extract. Furthermore, the tissue, which is collected from the biological samples and isolated as paraffin or cryostat section, can be used.

8. Screening Method of Therapeutic Agent of Myocardial Necrosis Using Periostin Variant (ΔbΔe) Proteins, DNA Encoding the Protein, or Antibody Recognizing Any of the Proteins

The protein to be used in the screening method of the present invention may include a periostin variant (ΔbΔe). The protein may be a naturally occurring protein or a protein produced by genetic engineering technique as long as it is a protein having an activity of the periostin variant (ΔbΔe), and an example of the naturally occurring protein may include a periostin variant (ΔbΔe) derived from any mammalian such as human, monkey, pig, cow, sheep, horse, rat, and mouse.

Specific examples of the periostin variant (ΔbΔe) may include a polypeptide having amino acid sequences of SEQ ID NOs: 2, 3, 5, 6, 8, and 9.

The DNA to be used in the screening method of the present invention may include a DNA encoding a periostin variant (ΔbΔe). Any DNAs may be used as long as they encode a protein to be used in the above-mentioned screening method of the present invention.

Specific examples of the DNA encoding a periostin variant (ΔbΔe) may include a DNA having nucleotide sequences of SEQ ID NO: 1, SEQ ID NO: 4, and SEQ ID NO: 7.

An antibody to be used in the screening method of the present invention may include an antibody recognizing the above-mentioned protein to be used in the screening method of the present invention or a polypeptide fragment thereof.

(1) Screening of Compound Specifically Acting on Periostin Variant (ΔbΔe) Protein

A microorganism, animal cells, or insect cells transformed to express a periostin variant (ΔbΔe) protein, and the purified periostin variant (ΔbΔe) protein are useful for screening a compound that specifically acts on the periostin variant (ΔbΔe). A compound obtained by screening is useful as a therapeutic agent of myocardial necrosis.

One of the methods of the above-mentioned screening is to select a compound that specifically induces the activation of a periostin variant (ΔbΔe) in an animal cell that has been transformed to produce a periostin variant (ΔbΔe) protein (hereinafter, referred to as “transformant for search”). The method for detecting the activation of the periostin variant (ΔbΔe) includes a method for measuring a cell response of the transformant for search.

Specific examples of the cell response may include, as an indicator, (1) cell migration activity, (2) phosphorylation of FAK (Focal Adhesion Kinase), (3) production of collagen, and the like.

In the above-mentioned (1), an effect of periostin on the cell migration ability of a primary cultured mouse myocardial fibroblast is assayed by, for example, an in-vitro cell migration assay. At this time, as the periostin, a purified protein, or a culture medium secreted from a transformant (transfectant) obtained by transferring an expression vector to a cell can be used. Since the periostin variant has a cell migration activity, the activity thereof can be assayed by using the cell migration activity.

In the above-mentioned (2), for example, a periostin protein is added in a serum-free cell culture solution of a mouse fetal-derived cell (for example, C3H10T1/2) and treated for one hour, and phosphorylation of FAK as the effect of the periostin on the cell is detected by Western blotting. The periostin variant is bound to integrin on the cell to activate the cell, and the cell response can be assayed based on the degree of the phosphorylation of FAK.

In the above-mentioned (3), for example, a periostin gene is introduced into a mouse fetal-derived cell (for example, C3H10T1/2), a mouse osteoblast-like cell (for example, MC3T3-E1), and a mouse periodontal membrane cell line (for example, A9) to form a transformant, and the production of collagen by the transformant is measured by using a commercially available kit (for example, Sircol Collagen Assay Kit; Funakoshi). Furthermore, the amount of cross-linked collagen can be assayed by using the same three types of cells, and treating these cells with the periostin, that is, a purified protein or a culture medium secreted from a transformant obtained by transferring an expression vector to a cell.

Furthermore, a purified periostin variant (ΔbΔe) protein or polypeptide constituting a part of the protein can be used for selecting a target compound that specifically binds to the periostin variant (ΔbΔe) protein. For example, the protein is immobilized to a solid phase carrier and the like, and tested sample is brought into contact with the carrier, the plate is sufficiently washed, and a compound bound to the periostin variant (ΔbΔe) protein is liberated from the protein. Thus, a target compound can be selected.

Another method of the above-mentioned screening is a method of efficiently screening a compound or protein, which selectively binds to the peptide, in which a large number of peptides constituting a part of the periostin variant (ΔbΔe) protein are synthesized on the plastic pin or a certain solid support medium with a high density (WO84/03564).

(2) Screening Method of Compound for Modulating Transcription or Translation of DNA Encoding Periostin Variant (ΔbΔe)

A compound having an activity of promoting an expression of mRNA of a periostin variant (ΔbΔe) gene or a periostin variant (ΔbΔe) protein in a heart-derived primary cultured cell or differentiation-induced cardiomyocyte is also useful as a therapeutic agent for myocardial necrosis.

The expression levels of mRNA of the periostin variant (ΔbΔe) gene in the heart-derived primary cultured cell (including cardiomyocyte and cardiac fibroblast) or differentiation-induced cardiomyocyte (for example, cardiomyocyte differentiation-induced from a precursor cell such as a bone marrow cell, cardiomyocyte differentiation-induced from an ES cell, and cardiomyocyte differentiation-induced from a pluripotent stem cell that has been differentiation-induced from a somatic cell) are measured and compared between the case where the above-mentioned cells are brought into contact with various test samples and the case where the above-mentioned cells are not brought into contact with the test samples. Thereby, a material for suppressing or promoting the transcription of the periostin variant (ΔbΔe) gene can be screened. The expression level of mRNA of the periostin variant (ΔbΔe) gene can be detected by the PCR method, Northern blotting, and RNase protective assay described in the above-mentioned 3.

The expression levels of mRNA of the periostin variant (ΔbΔe) protein in the heart-derived primary cultured cell or differentiation-induced cardiomyocyte are measured and compared between the case where the above-mentioned cells are brought into contact with various test samples and the case where the above-mentioned cells are not brought into contact with the test samples. Thereby, a material for suppressing or promoting of the transcription or translation of a periostin variant (ΔbΔe) gene can be screened. The expression level of the periostin variant (ΔbΔe) protein can be measured by a fluorescent antibody method, an enzyme-linked immunosorbent assay (ELISA), radioimmunoassay (RIA), an immunohistochemistry staining method (ABC method, CSA method, and the like) such as an immunohistological staining method and an immunocytological staining method, a Western blotting method, a dot blotting method, immunoprecipitation, sandwich ELISA, and an immunoagglutination method, which use an antibody specifically recognizing the periostin variant (ΔbΔe) protein mentioned in the above-mentioned 4.

The compound obtained by the above-mentioned method is administered as a drug to model animals with myocardial infarction such as ishemmia-reperfusion, in which ligation of the left ventricle descending coronary artery ligation (LAD ligation) is carried out and then reperfusion is carried out. The degree of healing and degree of suppression and the like of the myocardial necrosis site of the animals are determined by the well-known methods. Thus, it is possible to evaluate the therapeutic effect of the compound on the myocardial infarction.

9. Medicament for Treating and/or Preventing Myocardial Necrosis, which Contains Periostin Variant (ΔbΔe) Protein as Active Ingredient

A periostin variant (ΔbΔe) protein or a modified protein thereof can be used for healing the necrosis site or suppressing the reduction of the cardiac function by promoting the healing in various diseases with myocardial necrosis. Examples of the diseases with myocardial necrosis may include well-known diseases such as myocardial infarction and myocarditis (for example, viral myocarditis), and cardiomyopathy (for example, dilated cardiomyopathy).

In the medicament of the present invention, the above-mentioned periostin variant (ΔbΔe) protein or the modified protein thereof may be a naturally occurring protein or may be a recombinant protein produced by genetic engineering technique. Examples of the naturally occurring periostin variant (ΔbΔe) protein include a periostin variant (ΔbΔe) derived from any mammalians such as human, monkey, pig, cow, sheep, horse, mouse, rat, and mouse. When it is used as a medicament for treating and/or preventing human myocardial necrosis, that is, a cardiac function improving agent and a cardiac regeneration promoting agent, it is preferable that a protein whose amino acid sequence corresponds to that of a human-derived periostin variant (ΔbΔe) is used.

Medicaments for treating myocardial necrosis, which contain a periostin variant (ΔbΔe) protein as an active ingredient, may contain only the protein as an active ingredient but may contain other periostin variants or full-length periostin together. Furthermore, in general, it is desirable to provide pharmaceutical preparations produced by mixing one or more pharmacologically acceptable carriers by any methods that are well known in the technical field of pharmaceutics. Preferably, an aseptic solution such as an aqueous carrier such as aqueous solution of water or saline, glycine, glucose, human albumin, or the like, are used. Furthermore, in order to allow a preparation solution to approach the physiological conditions, pharmacologically acceptable additives such as a buffer agent or an isotonizing agent may be added. Examples of such additives may include sodium acetate, sodium chloride, sodium lactate, potassium chloride, sodium citrate, and the like. Furthermore, the preparations can be lyophilized and stored, and dissolved in an appropriate solution in use.

Desirable routes of administration are those that are the most effective for treatment, and include oral administration and parenteral administration such as intraoral, tracheobronchial, intrarectal, subcutaneous, intramuscular, and intravenous administrations. The dosage forms include aerosol, capsule, tablet, granule, syrup, emulsion, suppository, injection, ointment, tape, and the like.

Preparations suitable for oral administration include emulsion, syrup, capsule, tablet, powder, granule, and the like. For example, liquid preparations such as emulsion and syrup can be prepared by using, as an additive, water; sugars such as sucrose, sorbitol and fructose; glycols such as polyethylene glycol and propylene glycol; oils such as sesame oil, olive oil and soy bean oil; preservative such as p-hydroxybenzoic acid esters; flavors such as strawberry flavor and peppermint. Capsules, tablets, powders and granules can be produced by using, as an additive, excipient such as lactose, glucose, sucrose and mannitol; disintegrator such as starch and sodium alginate; lubricant such as magnesium stearate and talc; binder such as polyvinyl alcohol, hydroxypropylcellulose and gelatin; detergent such as fatty acid ester; and plasticizer such as glycerin.

Preparations suitable for parenteral administration include injection, suppository and aerosol. For example, an injection can be prepared by using a carrier including a salt solution, a glucose solution, or a mixture thereof. A suppository can be prepared by using a carrier such as cacao butter, hydrogenated oil, and carboxylic acid. Further, an aerosol can be prepared from the polypeptide itself or polypeptide with a carrier or the like that has no irritating effect on recipient's oral and airway mucous membrane and allows dispersion of the polypeptide as a fine particle to enhance the absorption thereof. Specific examples of such carriers are lactose and glycerin. Preparations such as aerosol and dry powder can be provided depending on the properties of the polypeptide and the carriers to be used. Further, the illustrated additives for the oral dosage forms can also be added as additives in these parenteral dosage forms.

Although the dosage and administration frequency depend on the intended treatment effect, a method of administration, a period of treatment, age, body weight, and the like, but it is typically within the range of 10 μg/kg/day to 10 mg/kg/day for an adult individual.

10. Medicament for Treating Myocardial Necrosis, which Contains Compound Obtained by Screening Method in 8 as Active Ingredient

A medicament for treating and/or preventing myocardial necrosis, which contains a compound obtained by a screening method in the above-mentioned 8 as an active ingredient, can be administered singly. Typically, however, it is desirable to provide it as a pharmaceutical preparation prepared by mixing the active ingredient with one or more pharmacologically acceptable carriers by an appropriate method that is well known in the art of pharmaceutics. Preferable pharmaceutical preparation forms and administration methods are described in the above-mentioned 9.

11. Gene Therapy Agent for Myocardial Necrosis, which Contains DNA Encoding Periostin Variant (ΔbΔe) Protein

Examples of a method for using a DNA encoding a periostin variant (ΔbΔe) protein as a gene therapy agent for myocardial necrosis include a method for preparing, formulating and administering the DNA singly or the DNA that has been inserted into an appropriate vector such as a retrovirus vector, an adenovirus vector, and an adenovirus associated virus vector according to usual methods described in the above-mentioned 9. Alternatively, the DNA may be administered by a non-viral method of gene transfer.

The recombinant virus vector can be constructed according to a method mentioned below. Based on the full-length cDNA of a periostin variant (ΔbΔe) protein, a DNA fragment having an appropriate length containing a portion encoding the protein is prepared if necessary. By inserting the DNA fragment or the full-length cDNA downstream of a promoter in a virus vector, a recombinant virus vector is constructed.

When the vector is an RNA virus vector, the recombinant virus can be created by preparing an RNA fragment homologous to the full-length cDNA of a periostin variant (ΔbΔe) and inserting it downstream of the promoter in the virus vector. The RNA fragment may be selected from a sense strand or an antisense strand, in addition to a double-stranded strand, depending on the type of the viral vector. For example, when a retroviral vector is used, an RNA that is homologous to the sense strand is selected. When a sense viral vector is used, an RNA that is homologous to the antisense strand is selected.

The recombinant virus vector is introduced into a packaging cell compatible with the vector. The packaging cells may be any of cells capable of supplying deleted proteins in the recombinant virus vector in which at least one of the genes encoding the proteins required for virus packaging is deleted. For example, human kidney-derived HEK293 cell, mouse fibroblast cell NIH3T3, or the like, may be used. Proteins to be supplied by the packaging cell include: mouse retrovirus-derived gag, pol, env and the like when using a retroviral vector; HIV virus-derived gag, pol, env, vpr, vpu, vif, tat, rev, nef and the like when using a lentiviral vector; adenovirus-derived E1A, E1B and the like when using an adenoviral vector; and Rep (p 5, p 19, and p 40), Vp (Cap) and the like when using an adeno-associated virus.

Virus vectors include those that can produce recombinant viruses in the above-mentioned packaging cells and have a promoter at a position capable of the transcription of a DNA encoding a periostin variant (ΔbΔe) in the target cells. Plasmid vectors include MFG [Proc. Natl. Acad. Sci. USA, 92, 6733-6737 (1995)], pBabePuro [Nucleic Acids Res., 18, 3587-3596 (1990)], LL-CG, CL-CG, CS-CG, CLG [Journal of Virology, 72, 8150-8157 (1998)], pAdex1 [Nucleic Acids Res., 23, 3816-3821 (1995)], and the like. As a promoter, any promoter can be used as long as it can function in human tissues, and examples of the promoter include a promoter of IE (immediate early) gene of Cytomegalovirus (human CMV), an early promoter of SV 40, a retroviral promoter, a metallothionein promoter, a heat-shock protein promoter, an SR α promoter, and the like. Further, an enhancer of the IE gene of the human CMV may be used along with the promoter.

Methods for introducing the recombinant virus vector into the packaging cells include, for example, a calcium phosphate method (Japanese Patent Application Laid-Open No. 2-227075) and a lipofection method (Proc. Natl. Acad. Sci. USA, 84, 7413 (1987)).

As a method for administering the above-mentioned recombinant virus vector, in addition to the method described in the above-mentioned 9, a virus vector can be oriented to the heart lesions by combination with a method of direct in vivo gene transfer using liposome delivery.

A virus vector can be prepared by combining an appropriately-sized DNA encoding a periostin variant (ΔbΔe) with a polylysine-conjugated specific antibody to the adenoviral hexon protein, and binding the resulting complex with the adenoviral vector. The virus vector reaches the target cells stably, is incorporated into the cell via an endosome, and disassembled in the cell. Thus, the vector can express a gene efficiently.

A DNA of a periostin variant (ΔbΔe) gene can be delivered to the lesions by a non-viral method of gene transfer.

Such non-viral methods of gene transfer known to those skilled in the art may include the calcium phosphate co-precipitation method [Virology, 52, 456-467 (1973); Science, 209, 1414-1422 (1980)], microinjection [Proc. Natl. Acad. Sci. USA, 77, 5399-5403 (1980); Proc. Natl. Acad. Sci. USA, 77, 7380-7384 (1980); Cell, 27, 223-231 (1981); Nature, 294, 92-94 (1981)], membrane fusion-mediated transfer using liposome [Proc. Natl. Acad. Sci. USA, 84, 7413-7417 (1987); Biochemistry, 28, 9508-9514 (1989); J. Biol. Chem., 264, 12126-12129 (1989); Hum. Gene Ther., 3, 267-275, (1992); Science, 249, 1285-1288 (1990); Circulation, 83, 2007-2011 (1992)], direct DNA incorporation and receptor-mediated DNA transfer method [Science, 247, 1465-1468 (1990); J. Biol. Chem., 266, 14338-14342 (1991); Proc. Natl. Acad. Sci. USA,_{—}87, 3655-3659 (1991); J. Biol. Chem., 264, 16985-16987 (1989); BioTechniques, 11, 474-485 (1991); Proc. Natl. Acad. Sci. USA, 87, 3410-3414 (1990); Proc. Natl. Acad. Sci. USA, 88, 4255-4259 (1991); Proc. Natl. Acad. Sci. USA, 87, 4033-4037 (1990); Proc. Natl. Acad. Sci. USA, 88, 8850-8854 (1991); Hum. Gene Ther., 3, 147-154 (1991)], or the like.

A study of tumor reports that the membrane fusion-mediated transfer method with the use of liposome allows local incorporation and expression of a gene by a tissue by administering a liposome preparation directly into the target tissue [Hum. Gene Ther. 3, 399-410 (1992)]. Thus, a similar effect may be expected for heart lesions. The technique of direct DNA incorporation to heart lesions is preferable to direct delivery of a DNA to the heart lesions. A receptor-mediated DNA transfer can be carried out, for example, with a protein ligand conjugated with the DNA (which is normally present as a covalently-closed super-coiled plasmid) via polylysine. The ligand is selected based on the corresponding ligand receptor expressed on the surface of a target cell or tissue. If desired, such a ligand-DNA conjugate can be injected directly to the blood vessel to reach a target tissue where the receptor binding and DNA-protein complex internalization occur. In order to prevent intracellular degradation of DNA, adenoviruses are co-infected with the DNA to disrupt the function of endosomes.

### EXAMPLES

Hereinafter, Examples specifically describe the present invention, but they do not limit the scope of the present invention.

### Example 1

### Investigation of Expression of Various Periostin Variants in Infarct Region after Acute Myocardial Infarction

In order to investigate the expression of a periostin variant in an infarct region after myocardial infarction, after C57BL/6 mice were allowed to have acute myocardial infarction (on Day 0, 1, 2, 3, 4, 5, 6, 7, 14, and 28), mRNA in the infarct region was purified and RT-PCR was carried out. The PCR product was analyzed by electrophoresis. Acute myocardial infarction was induced by ligating the left ventricle descending coronary artery according to the method described in Example 2 (3).

As shown in FIG. 1, periostin has various variants at the C-terminal, and each variant was thought to be correlated to the function of periostin. Therefore, the present inventors focused on the C-terminal and designed the below-mentioned primers so that

a periostin variant (Δb): splicing occurs in the position of the region b,

a periostin variant (Δe): splicing occurs in the position of the region e,

a periostin variant (ΔbΔe): splicing occurs in the positions of the regions b and e, and

a full-length periostin: splicing does not occur could be determined. Note here that in SEQ ID NO: 12 [cDNA sequence (including a signal) of mouse full-length periostin], the nucleotide numbers 1900 to 1968 are designated as a regional, nucleotide numbers 1969 to 2013 as a region a2, nucleotide numbers 2014 to 2094 as a region b, nucleotide numbers 2095 to 2184 as a region c1, nucleotide numbers 2185 to 2274 as a region c2, nucleotide numbers 2275 to 2352 as a region d, nucleotide numbers 2353 to 2436 as a region e, nucleotide numbers 2437 to 2478 as a region f1, and nucleotide numbers 2479 to 2514 as a region f2.

SEQ ID NO: 16:
P1F 5′-gataaaatacatccaaatcaagtttgttcg-3′
SEQ ID NO: 17:
P1R 5′-cgtggatcacttctggtcaccgtttcgc-3′
SEQ ID NO: 18:
P2F 5′-ctgaaaaacagactcgggaagaacg-3′
SEQ ID NO: 19:
P2R 5′-aaactctgtggtctggcctctggg-3′
SEQ ID NO: 20:
P3F 5′-gataaaatacatccaaatcaagtttgttcg-3′
SEQ ID NO: 21:
P3R 5′-aaactctgtggtctggcctctggg-3′
SEQ ID NO: 22:
gapdhF 5′-actttgtcaagctcatttcc-3′
SEQ ID NO: 23:
gapdhR 5′-tgcagcgaactttarrgctg-3′

The presence or absence of splicing in the region b can be determined by using a combination of the above-mentioned primer P1F and primer P1R (primer set 1). In FIG. 1, in a band detected at 236 bp, splicing does not occur at the position of b, and in a band detected at 155 bp, splicing occurs at the position of b.

The presence or absence of splicing in the region e can be determined by using a combination of the above-mentioned primer P2F and primer P2R (primer set 2). In a band detected at 325 bp, splicing does not occur at the position of e, and in a band detected at 241 bp, splicing occurs at the position of e.

The presence or absence of concurrent splicing in the region b and the region e can be determined by using a combination of the above-mentioned primer P3F and primer P3R (primer set 3). In a band detected at 685 bp, splicing occurs at neither the position b nor e, and in a band detected at 493 bp, splicing occurs concurrently at the positions b and e. Note here that in a band thinly detected between 493 bp and 685 bp, splicing occurs in the position b or e.

As a control, the expression of GAPDH was confirmed by using a combination of a primer gapdh F and a primer gapdh R.

As shown in FIG. 1, on Day 3 and 4 after acute myocardial infarction, only a periostin variant (ΔbΔe) including splicing in the positions b and e was expressed. Next, on Day 5 and 7, in addition to the periostin variant (ΔbΔe), the expression of a periostin variant (Δb) or a periostin variant (Δe) was slightly increased. On Day 28, the expression of the periostin variant (ΔbΔe) was reduced, and the expression of the periostin variant (Δb) or the periostin variant (Δe) or full-length periostin was increased.

Thus, in the process recovering from acute myocardial infarction, the function of each splice variant is different, and it is predicted that the periostin variant (ΔbΔe) plays an important role in particularly at the initial stage of recovery from injury.

### Example 2

### Creation and Property Observation of Periostin Knockout Mouse

In this Example, a periostin knockout mouse was created by Cre recombination according to the method described in Kitajima et al, 2000 development. 127: 3215-3226. Specific procedures are as follows.

### (1) Construction of Targeting Vector

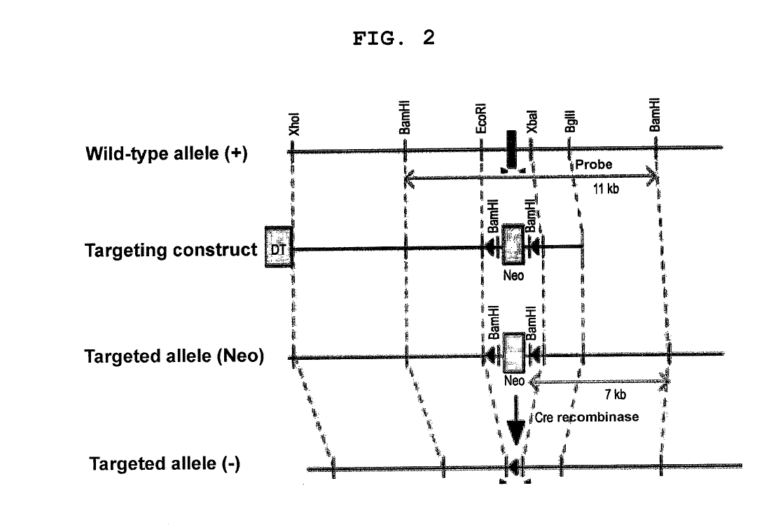

A BAC clone including exon 1 of a periostin gene was isolated from a mouse C57BL6/J BAC library according to a conventional method. FIG. 2 shows steps of targeting. Firstly, two homologous gene fragments, that is, 7.3 kb (XhoI-EcoRI) fragment and 1.2 kb (XbaI-BglII) fragment were subcloned to a PGK-Neo-PGK-DT-A cassette. A linear vector (50 μg) was subjected to electroporation to a TT2 ES cell by a method described in Yagi et al., 1993 Anal Biochem. 214: 70-76. From it, two of the G418-resistant ES clones were selected (#51 and #1051), and PCR was carried out by using a neo specific primer PGK-R and a periostin genomic primer Peri-R4 so as to confirm whether recombination occurred. In addition, these clones were confirmed by also a Southern blotting method according to a conventional method.

These investigations showed that a targeting vector for creating a periostin knockout mouse was constructed.

SEQ ID NO: 24:
PGK-R 5′-CTAAAGCGCATGCTCCAGACT-3′
SEQ ID NO: 25:
Peri-R4 5′-GCACCTGCCTCTTCCCAATTACAGG-3′

### (2) Creation of Periostin Knockout Mouse

Next, a chimera mouse was created by a coagulation method according to the method described in Kitajima et al, 2000 Development. 127: 3215-3226. The germ line of chimera was produced by using the above-produced ES cell clone (#51 or #1051). It was bred to an ICR mouse by high contribution of TT2 gene background (monitored by an agouti coat pigment). The #51 chimera mouse was mated with a CAG-Cre mouse described in Sakai and Miyazaki, 1997 Biochem. Biophys. Res. Commun. 237:318-324 in order to remove a neo cassette, and the established mouse thereof was bred to a C57BL/6 mouse. As a result, a mouse having an allele lacking a periostin gene was back-crossed in a C57BL6/J mouse for at least six generations.

In order to examine the genotypes of periostin +/+ mice (periostin wile-type mice) and periostin −/− mice (periostin knockout mice), PCR was carried out by using a specific primer in an intron in the periostin gene. For detecting a periostin wild-type mouse, a combination of the primer Wild-F and the primer Wild-R was used. For detecting a periostin knockout mouse, a combination of the primer Nock-F and the primer Nock-R was used.

From the investigation, it was confirmed that a knockout mouse was created.

SEQ ID NO: 26:
Wild-F 5′-gttcttacagaaagcagaaggatac-3′
SEQ ID NO: 27:
Wild-R 5′-ttaaatcactccacagcagaacacg-3′
SEQ ID NO: 28:
Nock-F 5′-catgatagcttctctcccagttctc-3′
SEQ ID NO: 29:
Nock-R 5′-cttgcaataagtaaaacagctcccc-3′

### (3) Property Observation of Periostin Knockout Mouse

The property of the periostin knockout mice created as mentioned above was investigated. The embryogenesis of the periostin knockout mice was apparently normal. After birth, the periostin knockout mice appeared to be healthy including fecundity except the eruption of cutting teeth. In addition, these mice survived for two or three weeks or more. Furthermore, when the developing heart was investigated, in 8-week-old or 10-week old mice, no cardiomyocyte abnormality was found in the myocardium, ventricular motion, valve function, pulsation, and blood pressure. Thus, also in the adult myocardium, significant symptoms were not found.

Then, the above-mentioned knockout mice were subjected to ligation of the left ventricle descending coronary artery to induce acute myocardial infarction (LAD ligation method). The above-mentioned operation was carried out according to the method described in Michael, 1995 Am J Physiol Heart Circ Physiol. 269: H2147-2154. Specific procedures are as follows. Under anesthesia, an 8 week-old mouse was intubated, and fixed to a respirator for rodents (SAR-830AP, manufactured by CWE). Moderate thoracotomy was carried out so as to select the left ventricle descending coronary artery. A 7-0 nylon suture was tied around the artery. The infarction was apparently observed from the change of colors of the left ventricle (LV). Finally, thoracotomy incision was closed. Only surviving mice were subjected to physiological measurement, and histological and biological analyses. By using a middle part of the heart (mid-part) section from at least five mice, the infarct size was determined and a region with a risk of infarction was examined according to the method described in the above-mentioned document (Michael et al, 1995).

There was no significant difference in body weight and heart rate among periostin −/− mice (periostin knockout mice), periostin +/− mice (periostin chimera mice), and periostin +/+ mice (periostin normal mice) in a normal control state and after acute myocardial infarction. Furthermore, on Day 1, 7, and 28 after acute myocardial infarction, there was no difference in the infarct size between the periostin knockout mice and the periostin normal mice. These results are shown in Table 1.

As to the measurement items in Table 1, BW denotes a body weight (g). Hereinafter, similarly, HR denotes a heart rate (beat/min), LVEDD denotes a left ventricular end-diastolic dimension (mm), LVESD denotes a left ventricular end-systolic dimension (mm), AW denotes an anterior wall thickness (mm), PW denotes a posterior wall thickness (mm), FS denotes left ventricular diameter shortening rate (%), and IS denotes a infarct size (%), respectively. Furthermore, the reference mark “a” in Table 1 shows P<0.05 with respect to periostin +/+ mice in which acute myocardial infarction is induced.

TABLE 1

| | Control | | MI day 1 | | MI day 7 | | MI day 28 | |
|---|---|---|---|---|---|---|---|---|
| Periostin | +/+ | −/− | +/+ | −/− | +/+ | −/− | +/+ | −/− |
| BW (g) | 20.4 ± 0.3 | 20.3 ± 0.3 | 20.4 ± 0.5 | 20.4 ± 0.6 | 20.1 ± 0.3 | 19.7 ± 0.4 | 24.3 ± 0.4 | 24.3 ± 0.5 |
| HR (beats/min) | 458 ± 14 | 461 ± 9 | 425 ± 14 | 415 ± 14 | 397 ± 23 | 408 ± 15 | 458 ± 12 | 461 ± 21 |
| LVEDD (mm) | 3.44 ± 0.05 | 3.41 ± 0.03 | 3.82 ± 0.06 | 3.82 ± 0.06 | 4.66 ± 0.19 | 4.15 ± 0.03^{a} | 5.19 ± 0.17 | 4.48 ± 0.23^{a} |
| LVESD (mm) | 1.76 ± 0.03 | 1.73 ± 0.02 | 2.88 ± 0.08 | 2.88 ± 0.08 | 3.66 ± 0.24 | 3.09 ± 0.04^{a} | 4.47 ± 0.20 | 3.53 ± 0.29^{a} |
| AW (mm) | 0.59 ± 0.01 | 0.58 ± 0.00 | 0.59 ± 0.03 | 0.59 ± 0.04 | 0.49 ± 0.01 | 0.50 ± 0.01 | 0.45 ± 0.01 | 0.48 ± 0.02 |
| PW (mm) | 0.60 ± 0.01 | 0.58 ± 0.00 | 0.58 ± 0.01 | 0.57 ± 0.01 | 0.61 ± 0.01 | 0.59 ± 0.03 | 0.66 ± 0.02 | 0.63 ± 0.02 |
| FS (%) | 48.9 ± 0.6 | 49.2 ± 0.3 | 24.7 ± 1.0 | 24.6 ± 1.5 | 21.9 ± 2.4 | 25.6 ± 0.9 | 14.3 ± 1.5 | 22.1 ± 2.9^{a} |
| IS (%) | — | — | 50.9 ± 2.4 | 51.1 ± 3.7 | 50.1 ± 2.7 | 49.4 ± 3.3 | 46.8 ± 2.1 | 45.5 ± 2.4 |

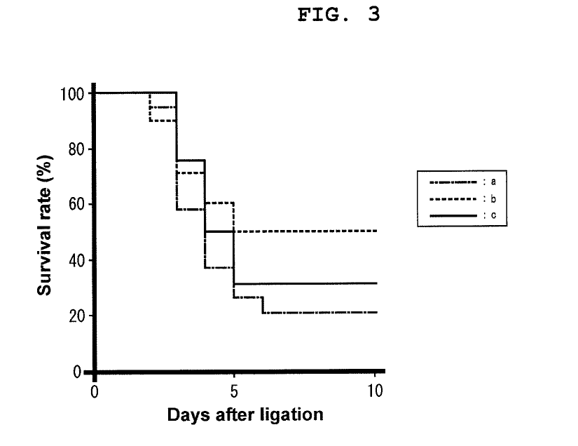

On the other hand, after acute myocardial infarction, the survival rate of periostin knockout mice was extremely lower than that of periostin normal mice. The survival rate of periostin chimera mice was the same level as that of periostin normal mice. These results are shown in FIG. 3. From the results, cardiac rupture was significantly observed in the periostin knockout mice. It was clarified that in the knockout mice, the infarct region was not healed and the tissue became mechanically weak due to lack in periostin, resulting in easily inducing cardiac rupture.

### Example 3

### Expression of Periostin Variant (ΔbΔe) to Periostin Knockout Mouse

(1) Production of Adenovirus for Expression of Periostin Variant (ΔbΔe)

By using a commercially available expression kit (Adeno-X expression system2; manufactured by BD Bioscience), an Ad-ΔbΔe vector was constructed as a periostin variant (ΔbΔe) expression adenovirus vector, an Ad-Full vector was constructed as a full-length periostin expression virus vector, and an Ad-nlsLacZ vector was constructed for control, respectively. As a cDNA encoding a periostin variant (ΔbΔe) protein, a cDNA consisting of a nucleotide sequence of SEQ ID NO: 4 was used, and as a cDNA encoding a full-length periostin, a cDNA consisting of a nucleotide sequence of SEQ ID NO: 12 was used.

Purification of virus was carried out by a cesium chloride method according to a method described in Ugai, 2005 BBRC. 331:1053-1060.

### (2) Expression and Property Observation of Periostin Variant (ΔBΔE) in Periostin Knockout Mouse

One day before acute myocardial infarction (by LAD ligation method) was induced, a solution (1.6×10¹⁰ pfu, 100 μL) including the above-produced virus (Ad-ΔbΔe virus, Ad-Full virus, or Ad-nlsLacZ virus) was administered in a periostin knockout mouse by caudal vein injection.

After acute myocardial infarction was induced, the properties of periostin knockout mice to which respective viruses were administered were observed. When the Ad-ΔbΔe virus was administered to the periostin knockout mice, cardiac rupture was recovered to about 50%. On the other hand, when the Ad-Full virus was administered, it was recovered to about 30%, and when the Ad-nlsLacZ virus was administered, it was recovered to only about 20%.

With the full-length periostin, neither the degree of cardiac rupture nor the healing of infarction was improved. However, with the periostin variant (ΔbΔe), both were significantly ameliorated. This clearly showed that in particular, a periostin variant (ΔbΔe) plays an important role in recovering from acute myocardial infarction.

### INDUSTRIAL APPLICABILITY

The present invention can be used for treating or preventing diseases with myocardial necrosis, and furthermore for testing or monitoring diseases with myocardial necrosis.

In the above, the present invention has been described along certain embodiments. Nevertheless, it will be understood that various alteration and modifications obvious to a person skilled in the art are in the scope of the present invention.

### Sequence Listing Free Text

Each nucleotide sequence of SEQ ID NOs: 16 to 29 in the sequence listing shows a primer sequence, specifically, primer P1F (SEQ ID NO: 16), primer P1R (SEQ ID NO: 17), primer P2F (SEQ ID NO: 18), primer P2R (SEQ ID NO: 19), primer P3F (SEQ ID NO: 20), primer P3R (SEQ ID NO: 21), primer gapdhF (SEQ ID NO: 22), primer gapdhR (SEQ ID NO: 23), primer PGK-R (SEQ ID NO: 24), primer Peri-R4 (SEQ ID NO: 25), primer Wild-F (SEQ ID NO: 26), primer Wild-R (SEQ ID NO: 27), primer Nock-F (SEQ ID NO: 28), and primer Nock-R (SEQ ID NO: 29).

1. A medicament for treating or preventing diseases with myocardial necrosis, comprising a periostin variant (ΔbΔe) polypeptide as an active ingredient.

2. A medicament for treating or preventing diseases with myocardial necrosis, comprising, as an active ingredient:
[a] a polypeptide comprising the amino acid sequence of SEQ ID NO: 3, SEQ ID NO: 6, or SEQ ID NO: 9;
[b] a polypeptide comprising an amino acid sequence in which one or several amino acids are deleted, substituted or added in the amino acid sequence of SEQ ID NO: 3, SEQ ID NO: 6, or SEQ ID NO: 9, and having an activity of healing a myocardial necrosis site; or
[c] a polypeptide comprising an amino acid sequence that has an identity of not less than 60% to the amino acid sequence of SEQ ID NO: 3, SEQ ID NO: 6, or SEQ ID NO: 9, and having an activity of healing a myocardial necrosis site.

3. The medicament according to claim 1, wherein the medicament has an effect of ameliorating a cardiac function.

4. The medicament according to claim 1, wherein the medicament has an effect of promoting cardiac regeneration.

5. A method for treating or preventing diseases with myocardial necrosis, the method comprising administering a therapeutically effective amount of periostin variant (ΔbΔe) polypeptide to a subject to be treated.

6. A method for treating or preventing diseases with myocardial necrosis, the method comprising administering a therapeutically effective amount of polypeptide to a subject to be treated, the polypeptide being selected from the group consisting of:
[a] a polypeptide comprising the amino acid sequence of SEQ ID NO: 3, SEQ ID NO: 6, or SEQ ID NO: 9;
[b] a polypeptide comprising an amino acid sequence in which one or several amino acids are deleted, substituted or added in the amino acid sequence of SEQ ID NO: 3, SEQ ID NO: 6, or SEQ ID NO: 9, and having an activity of healing a myocardial necrosis site; or
[c] a polypeptide comprising an amino acid sequence that has an identity of not less than 60% to the amino acid sequence of SEQ ID NO: 3, SEQ ID NO: 6, or SEQ ID NO: 9, and having an activity of healing a myocardial necrosis site.

7. The method according to claim 5, wherein the method has an effect of ameliorating a cardiac function.

8. The method according to claim 5, wherein the method has an effect of promoting cardiac regeneration.

9-12. (canceled)

13. A medicament for treating or preventing diseases with myocardial necrosis, comprising a polynucleotide encoding a periostin variant (ΔbΔe).

14. A medicament for treating or preventing diseases with myocardial necrosis, comprising:
[a] a polynucleotide encoding a polypeptide comprising the amino acid sequence of SEQ ID NO: 3, SEQ ID NO: 6, or SEQ ID NO: 9;
[b] a polynucleotide encoding a polypeptide comprising an amino acid sequence in which one or several amino acids are deleted, substituted or added in the amino acid sequence of SEQ ID NO: 3, SEQ ID NO: 6, or SEQ ID NO: 9, and having an activity of healing a myocardial necrosis site;
[c] a polynucleotide encoding a polypeptide comprising an amino acid sequence having an identity of not less than 60% to the amino acid sequence of SEQ ID NO: 3, SEQ ID NO: 6, or SEQ ID NO: 9 and having an activity of healing a myocardial necrosis site; or
[d] a polynucleotide comprising a nucleotide sequence of SEQ ID NO: 1, SEQ ID NO: 4, or SEQ ID NO: 7.

15. The medicament according to claim 13, wherein the medicament has an effect of ameliorating a cardiac function.

16. The medicament according to claim 13, wherein the medicament has an effect of promoting cardiac regeneration.

17. A method for treating or preventing diseases with myocardial necrosis, the method comprising administering a therapeutically effective amount of polynucleotide encoding a periostin variant (ΔbΔe) to a subject to be treated.

18. A method for treating or preventing diseases with myocardial necrosis, the method comprising administering a therapeutically effective amount of polynucleotide to a subject to be treated, the polynucleotide being selected from the group consisting of:
[a] a polynucleotide encoding a polypeptide comprising the amino acid sequence of SEQ ID NO: 3, SEQ ID NO: 6, or SEQ ID NO: 9;
[b] a polynucleotide encoding a polypeptide comprising an amino acid sequence in which one or several amino acids are deleted, substituted or added in the amino acid sequence of SEQ ID NO: 3, SEQ ID NO: 6, or SEQ ID NO: 9, and having an activity of healing a myocardial necrosis site;
[c] a polynucleotide encoding a polypeptide comprising an amino acid sequence having an identity of not less than 60% to the amino acid sequence of SEQ ID NO: 3, SEQ ID NO: 6, or SEQ ID NO: 9 and having an activity of healing a myocardial necrosis site; or
[d] a polynucleotide comprising a nucleotide sequence of SEQ ID NO: 1, SEQ ID NO: 4, or SEQ ID NO: 7.

19. The method according to claim 17, wherein the method has an effect of ameliorating a cardiac function.

20. The method according to claim 17, wherein the method has an effect of promoting cardiac regeneration.

21-24. (canceled)

25. A diagnostic agent for diseases with myocardial necrosis, comprising:
[a] a DNA encoding a periostin variant (ΔbΔe) polypeptide, or a partial fragment thereof;
[b] a DNA consisting of a nucleotide sequence of SEQ ID NO: 1, SEQ ID NO: 4, or SEQ ID NO: 7, or a partial fragment thereof; or
[c] an oligonucleotide having a sequence consisting of consecutive 5 to 60 nucleotides in the nucleotide sequence of SEQ ID NO: 1, SEQ ID NO: 4 or SEQ ID NO: 7, or a derivative thereof, and an oligonucleotide having a sequence complementary to the oligonucleotide, or a derivative thereof.

26. A diagnostic agent for diseases with myocardial necrosis, comprising an antibody specifically recognizing a periostin variant (ΔbΔe) polypeptide.

27. A diagnostic method for diseases with myocardial necrosis, the method comprising the steps of:
(1) preparing a DNA or cDNA derived from a test specimen from a biological sample obtained from a subject to be tested;
(2) detecting a mutation of a DNA encoding a periostin variant (ΔbΔe) polypeptide in the DNA or cDNA derived from the test specimen, by using:
[a] the DNA encoding the periostin variant (ΔbΔe) polypeptide, or a partial fragment thereof;
[b] a DNA consisting of a nucleotide sequence of SEQ ID NO: 1, SEQ ID NO: 4, or SEQ ID NO: 7, or a partial fragment thereof; or
[c] an oligonucleotide having a sequence consisting of consecutive 5 to 60 nucleotides in the nucleotide sequence of SEQ ID NO: 1, SEQ ID NO: 4 or SEQ ID NO: 7, or a derivative thereof, and an oligonucleotide having a sequence complementary to the oligonucleotide, or a derivative thereof, and
(3) determining a risk, a type, a degree and/or a state of a disease with myocardial necrosis based on the mutation.

28. A diagnostic method for diseases with myocardial necrosis, the method comprising the steps of:
(1) preparing a DNA or cDNA derived from a test specimen from a biological sample obtained from a subject to be tested;
(2) specifically amplifying a DNA encoding a periostin variant (ΔbΔe) polypeptide in the DNA or cDNA derived from the test specimen, and analyzing an expression level thereof, by using:
[a] the DNA encoding the periostin variant (ΔbΔe) polypeptide, or a partial fragment thereof;
[b] a DNA consisting of a nucleotide sequence of SEQ ID NO: 1, SEQ ID NO: 4, or SEQ ID NO: 7, or a partial fragment thereof; or
[c] an oligonucleotide having a sequence consisting of consecutive 5 to 60 nucleotides in the nucleotide sequence of SEQ ID NO: 1, SEQ ID NO: 4 or SEQ ID NO: 7, or a derivative thereof, and an oligonucleotide having a sequence complementary to the oligonucleotide, or a derivative thereof, and
(3) determining a degree and/or a state of a disease with myocardial necrosis based on the expression level.

29. A diagnostic method for diseases with myocardial necrosis, the method comprising the steps of:
(1) preparing a specimen from a biological sample obtained from a subject to be tested;
(2) detecting an expression level and/or a structural change of a periostin variant (ΔbΔe) polypeptide in the specimen by using an antibody specifically recognizing the periostin variant (ΔbΔe) polypeptide, and
(3) determining a risk, a cause, a degree, and/or a state of a disease with myocardial necrosis based on the expression level and/or the structural change.

30. A screening method for a medicament for treating or preventing diseases with myocardial necrosis, wherein (i) an expression level of the polypeptide in a cell expressing the polypeptide described in claim 1 or 2 and (ii) an expression level of the polypeptide when the cell expressing the polypeptide is brought into contact with a testing material are compared with each other so as to select a material for increasing the expression level of the polypeptide.

31. A screening method for a medicament for treating or preventing diseases with myocardial necrosis, wherein (i) a function of a cell expressing the polypeptide described in claim 1 or 2 and (ii) a function of the cell when the cell expressing the polypeptide is brought into contact with a testing material are compared with each other so as to select a material having an activity of controlling the function of the cell.

32. The screening method according to claim 30, wherein the cell is a cell lacking a gene encoding periostin.

33. The method according to claim 30, wherein the medicament for treating or preventing diseases with myocardial necrosis is a material having an effect of ameliorating a cardiac function.

34. The method according to claim 30, wherein the medicament for treating or preventing diseases with myocardial necrosis is a material having an effect of promoting cardiac regeneration.

35. A compound obtained by the method according to claim 30, or a pharmacologically acceptable salt thereof.

36. A medicament for treating or preventing diseases with myocardial necrosis, comprising the compound or the pharmacologically acceptable salt thereof according to claim 35.

37. The medicament for treating or preventing diseases with myocardial necrosis according to claim 36, wherein the medicament has an effect of ameliorating a cardiac function.

38. The medicament for treating or preventing diseases with myocardial necrosis according to claim 36, wherein the medicament has an effect of healing and promoting cardiac regeneration.

39. A method for treating or preventing diseases with myocardial necrosis, the method comprising administering a therapeutically effective amount of the compound or the pharmacologically acceptable salt thereof according to claim 35 to a subject to be treated.

40. (canceled)

41. An antibody specifically recognizing a periostin variant (ΔbΔe) protein.

42. An immunological detection or a quantitation method of a periostin variant (ΔbΔe) protein, which uses the antibody according to claim 41.

43. The screening method according to claim 31, wherein the cell is a cell lacking a gene encoding periostin.

44. The method according to claim 31, wherein the medicament for treating or preventing diseases with myocardial necrosis is a material having an effect of ameliorating a cardiac function.

45. The method according to claim 31, wherein the medicament for treating or preventing diseases with myocardial necrosis is a material having an effect of promoting cardiac regeneration.

46. A compound obtained by the method according to claim 31, or a pharmacologically acceptable salt thereof.
